(19) United States
(12) Patent Application Publication
(10) Pub. No.: US 2014/0287024 A1
(43) Pub. Date: Sep. 25, 2014

(54) NANOPARTICLE-BASED DELIVERY SYSTEM WITH OXIDIZED PHOSPHOLIPIDS AS TARGETING LIGANDS FOR THE PREVENTION, DIAGNOSIS AND TREATMENT OF ATHEROSCLEROSIS

(71) Applicants: Shu WANG, Lubbock, TX (US); Guigen LI, Lubbock, TX (US); Zhaoyang FAN, Lubbock, TX (US)

(72) Inventors: Shu WANG, Lubbock, TX (US); Guigen LI, Lubbock, TX (US); Zhaoyang FAN, Lubbock, TX (US)

(21) Appl. No.: 14/198,481

(22) Filed: Mar. 5, 2014

Related U.S. Application Data
(60) Provisional application No. 61/775,420, filed on Mar. 8, 2013.

(51) Int. Cl. A61K 31/353; A61K 47/24; A61K 39/395; A61K 9/127

(52) U.S. Cl. CPC A61K 31/353; A61K 9/127; A61K 47/24; A61K 39/3955
USPC 424/450; 424/172.1; 514/456

(57) ABSTRACT

Disclosed are nanoparticle-based medicine/nutrient delivery system that are coated or incorporated with oxidized phospholipids as targeting ligands. Such delivery systems can specifically target macrophages, which are determinant cells in the aortic wall for atherosclerotic lesion development, to significantly increase bioavailability and specificity for the prevention, diagnosis and treatment of atherosclerosis.

### CROSS-REFERENCE TO RELATED APPLICATIONS

This application claims priority to U.S. provisional application No. 61/775,420, filed on Mar. 8, 2013, which is incorporated by reference herein in its entirety.

### STATEMENT OF GOVERNMENT SUPPORT

This invention was made with U.S. Government support under Grant No. R15-AT007013-01 and No. R21-DA031860-01 awarded by National Institute of Health (NIH). The U.S. Government has certain rights in the invention.

### FIELD

The invention disclosed herein generally relates to the field of using oxidized phospholipids as targeting ligands coated on nanoparticle-based medicine/nutrient delivery system. Such delivery systems can specifically target macrophages, which are determinant cells in the aortic wall for atherosclerotic lesion development, to significantly increase bioavailability and specificity for the prevention, diagnosis and treatment of atherosclerosis.

### BACKGROUND

Cardiovascular disease (CVD) is the No. 1 killer in the United States. Atherosclerosis is the major cause of CVD, accounting for over half the deaths attributed to CVD. The buildup of cholesterol in the aortic wall is called plaque, which is the hallmark event in the development of atherosclerosis. Atherosclerosis is a progressive disease characterized by lipid plaque formation in arteries. Macrophages play an important role in atherosclerotic lesion progression by facilitating cholesterol accumulation and increasing inflammatory responses in aortic walls (Kunjathoor et al., 2002; Ludewig and Laman, 2004). After monocytes chemoattractant protein-1 (MCP-1, a chemokine) and its receptor direct the migration of monocytes into the intima, those monocytes are differentiated to macrophages in the intimal layer of arterial wall. These macrophages take up cholesterol-rich low-density lipoprotein (LDL), leading to the formation of cholesterol-laden macrophages (foam cells), which characterize the early atherosclerotic lesion. In 1979, Nobel laureates Brown and Goldstein found that the rate of oxidized LDL (oxLDL) uptake and degradation was 20 times higher than that of native LDL in resident mouse peritoneal macrophages (Brown et al., 1979; Goldstein et al., 1979). They named the oxLDL binding site as the macrophage scavenger receptor for its role in scavenging modified LDL. Since then, scavenger receptors have drawn tremendous research attention. Macrophage scavenger receptor AI, AII and CD36 are major membrane proteins involved in the uptake of cholesterol-rich oxLDL (de Winther et al., 2000; Kunjathoor et al., 2002). Studies performed in mice lacking CD36 showed a very significant reduction (76.5%) in aortic lesion size, and peritoneal macrophages isolated from those mice exhibited a 60-80% decrease in both oxLDL binding and oxLDL uptake (Febbraio et al., 1999). This suggests that CD36-mediated oxLDL uptake is required for foam cell formation and lesion development during atherosclerosis (Febbraio et al., 2000; Moore and Freeman, 2006). CD36 correlates well with lesion severity (Curtiss, 2009; Febbraio et al., 2000; Moore and Freeman, 2006). CVD is known as a silent killer, because the lack of techniques for early detection and targeted prevention and treatment. Targeting CD36 is a promising avenue for diagnosis and targeted prevention and treatment of atherosclerosis (Berliner et al., 2009; Harb et al., 2009; Lipinski et al., 2009; Silverstein, 2009).

Currently, the techniques and methods of specifically targeting to the atherosclerotic lesion are not available. Therefore, the efficient and early diagnosis, prevention and treatment of atherosclerosis and related diseases are impossible.

What is needed in the art are methods and compositions that will improve the prevention, diagnosis and treatment of atherosclerosis and related diseases.

### SUMMARY

In one aspect, provided herein is a composition comprising a plurality of nanoparticles that comprises one or more oxidized phospholipids encapsulated within, adhered to a surface of, or integrated into the structure of the nanoparticles. The one or more oxidized phospholipids target an atherosclerotic lesion site; for example via binding to scavenger receptors on surfaces of macrophages. In some embodiments, the scavenger receptor is involved in the uptake of cholesterol-rich modified lipoproteins such as oxLDL. In some embodiments, the scavenger receptor is CD36.

In some embodiments, the nanoparticles are selected from the group consisting of liposomes, polymerosomes, microspheres, micro-structured lipid carriers, nano-structured lipid carriers, and a combination thereof.

In some embodiments, the one or more oxidized phospholipids are selected from the group consisting of 1-palmitoyl-2-azelaoyl-sn-glycero-3-phosphocholine, 1-hexadecyl-2-azelaoyl-sn-glycero-3-phosphocholine, 1-palmitoyl-2-glutaroyl-sn-glycero-3-phosphocholine, 1-palmitoyl-2-(5-oxovaleroyl)-sn-glycero-3-phosphocholine, and 1-palmitoyl-2-(9-oxononanoyl)-sn-glycero-3-phosphocholine, an ester of lysophosphatidylcholine, an ester of 1-lysophosphatidylcholine (1-lysoPC), an ester of 2-lysophosphatidylcholine (2-lysoPC1-hexadecyl-2-acetoyl-sn-glycero-3-phosphocholine, 1-octadecyl-2-acetoyl-sn-glycero-3-phosphocholine, 1-hexadecyl-2-butyroyl-sn-glycero-3-phosphocholine, 1-octadecyl-2-butyroyl-sn-glycero-3-phosphocholine, 1-palmitoyl-2-acetoyl-sn-glycero-3-phosphocholine, 1-octadecenyl-2-acetoyl-sn-glycero-3-phosphocholine, 1-hexadecyl-2-(homogammalinolenoyl)-sn-glycero-3-phosphocholine, 1-hexadecyl-2-arachidonoyl-sn-glycero-3-phosphocholine, 1-hexadecyl-2-eicosapentaenoyl-sn-glycero-3-phosphocholine, 1-hexadecyl-2-docosahexaenoyl-sn-glycero-3-phosphocholine, 1-octadecyl-2-methyl-sn-glycero-3-phosphocholine, 1-hexadecyl-2-butenoyl-sn-glycero-3-phosphocholine, Lyso PAF C16, Lyso PAF C18, 1-O-1′-(Z)-hexadecenyl-2-[12-[(7-nitro-2-1,3-benzoxadiazol-4-yl)-amino]dodecanoyl]-sn-glycero-3-phosphocholine, 1-O-1-(Z)-hexadecenyl-2-oleoyl-sn-glycero-3-phosphocholine, 1-O-1-(Z)-hexadecenyl-2-arachidonoyl-sn-glycero-3-phosphocholine, 1-O-1′-(Z)-hexadecenyl-2-docosahexaenoyl-sn-glycero-3-phosphocholine, 1-O-1-(Z)-hexadecenyl-2-oleoyl-sn-glycero-3-phosphoethanolamine, 1-O-1′-(Z)-hexadecenyl-2-arachidonoyl-sn-glycero-3-phosphoethanolamine, 1-O-1′-(Z)-hexadecenyl-2-docosahexaenoyl-sn-glycero-3-phosphoethanolamine, and a combination thereof.

In some embodiments, the one or more oxidized phospholipids are selected from the group consisting of 9-hydroxy-10-dodecenedioic acid esters of 2-lyso-PC (HDdiA-PC), 5-hydroxy-8-oxo-6-octenedioic acid esters of 2-lyso-PC (HOdiA-PC), 9-hydroxy-12-oxo-10-dodecenoic acid esters of 2-lyso-PC (HODA-PC), 5-hydroxy-8-oxo-6-octenoic acid esters of 2-lyso-PC (HOOA-PC), 9-keto-12-oxo-10-dodecenoic acid esters of 2-lyso-PC (KODA-PC), 5-keto-8-oxo-6-octenoic acid esters of 2-lyso-PC (KOOA-PC), 9-keto-10-dodecendioic acid esters of 2-lyso-PC (KDdiA-PC), and 5-keto-6-octendioic acid esters of 2-lyso-PC (KOdiA-PC), 5-oxovaleric acid esters of 2-lyso-PC (OV-PC), and 9-oxononanoic acid esters of 2-lyso-PC (ON-PC), 1-palmitoyl-2-(5-oxovaleroyl)-phosphatidylcholine (POVPC), and a combination thereof. In some embodiments, the one or more oxidized phospholipids comprise KOdiA-PC or KDdiA-PC.

In some embodiments, the nanoparticles consist of the one or more oxidized phospholipids. The one or more oxidized phospholipids are selected from the group consisting of 9-hydroxy-10-dodecenedioic acid esters of 2-lyso-PC (HDdiA-PC), 5-hydroxy-8-oxo-6-octenedioic acid esters of 2-lyso-PC(HOdiA-PC), 9-hydroxy-12-oxo-10-dodecenoic acid esters of 2-lyso-PC(HODA-PC), 5-hydroxy-8-oxo-6-octenoic acid esters of 2-lyso-PC(HOOA-PC), 9-keto-12-oxo-10-dodecenoic acid esters of 2-lyso-PC (KODA-PC), 5-keto-8-oxo-6-octenoic acid esters of 2-lyso-PC (KOOA-PC), 9-keto-10-dodecendioic acid esters of 2-lyso-PC (KDdiA-PC), and 5-keto-6-octendioic acid esters of 2-lyso-PC (KOdiA-PC), 5-oxovaleric acid esters of 2-lyso-PC (OV-PC), and 9-oxononanoic acid esters of 2-lyso-PC (ON-PC), 1-palmitoyl-2-(5-oxovaleroyl)-phosphatidylcholine (POVPC), and a combination thereof.

In some embodiments, the nanoparticles have an average linear dimension of between about 10 nanometers to about 2,500 nanometers.

In some embodiments, the nanoparticles comprise one or more molecules selected from the group consisting of albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(-hydroxyvalerate), poly(D,L-lactide-co-glycolide), poly(1-lactide-co-glycolide), poly(-hydroxybutyrate), poly(-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, apolioprotein A1, apolioprotein A2, other apolioproteins and a combination thereof.

In some embodiments, the nanoparticles comprise one or more molecules selected from the group consisting of gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, and a combination thereof.

In some embodiments, the nanoparticles comprise a biodegradable polymer comprises PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride), and a combination thereof.

In some embodiments, the nanoparticles are liposomes comprising phospholipids, cholesterol, sphingolipids, ceramides or hapten-conjugated lipids.

In one aspect, the nanoparticles further comprise an additional targeting ligand, encapsulated within, adhered to a surface of, or integrated into the structure of the nanoparticles. The additional targeting ligand also targets an atherosclerotic lesion site. In some embodiments, each of the nanoparticles comprises both the one or more oxidized phospholipids and the additional targeting ligand. In some embodiments, the additional targeting ligand is an antibody that recognizes a target (e.g., a protein) at or near an atherosclerotic lesion site. For example, the additional targeting ligand is and antibody or an antibody fragment that is reactive to one selected from the group consisting of oxidized LDL, scavenger receptor A (the first OxLDL receptor to be characterized and cloned, CD36, CD68, Lectin-like oxidized LDL receptor-1 (LOX-1), SR-A1, SR-B1, and a combination thereof.

In one aspect, the composition provided herein further comprises one or more bioactive agents that are encapsulated within, adhered to a surface of, or integrated into the structure of the nanoparticles. The one or more bioactive agents are delivered to or near an atherosclerotic lesion site.

In some embodiments, the one or more bioactive agents comprise a first bioactive agent that provides an indicium for the presence of the atherosclerotic lesion site. In some embodiments, the indicium is a fluorescent signal emitted upon binding of the first bioactive agent to or near the atherosclerotic lesion site. In some embodiments, the one or more bioactive agents further comprise: a second bioactive agent that exhibits a therapeutic effect on the atherosclerotic lesion site. In some embodiments, the one or more bioactive agents further comprise a first bioactive agent and a second bioactive agent.

In one aspect, the composition provided herein further comprises: an adjuvant or a pharmaceutically compatible carrier.

In one aspect, provided herein is a method of preventing, diagnosing and/or treating atherosclerosis in a patient. The method comprises administering an effective amount of a composition, to or near a known or suspected atherosclerotic lesion site. The composition comprises a plurality of nanoparticles comprising one or more oxidized phospholipids encapsulated within, adhered to a surface of, or integrated into the structure of the nanoparticles. The one or more oxidized phospholipids target the atherosclerotic lesion site.

In some embodiments, the administering step comprises intraarterial or intravenous delivery of the nanoparticles. In some embodiments, intraarterial or intravenous delivery comprises using a catheter. In some embodiments, intraarterial or intravenous delivery comprises direct injection.

One of skill in the art would understand that different embodiments of oxidized phospholipids disclosed herein and different embodiments of nanoparticles disclosed herein can be used in connection with any aspect described.

### BRIEF DESCRIPTION OF THE DRAWINGS

Those of skill in the art will understand that the drawings, described below, are for illustrative purposes only. The drawings are not intended to limit the scope of the present teachings in any way.

The present invention is of methods and compositions assembling nanoparticles with diagnostic and anti-atherosclerotic agents encapsulated and incorporated, with oxidized phospholipids coated on the surface as target ligands to increase their level of stability, cellular bioavailability and targeting to aortic intimal macrophages, with the goal of diagnosis, preventing and reversing atherosclerotic lesion development. References and figures with legends may help to better understand the principles and operation of the present invention.

It is to be understood that the invention is not limited in its applications to the details in the following description or examples, and the phraseology and terminology employed herein is for the purpose of description and explanation and should not be regarded as limiting.

FIG. 1 is an illustration of nanoparticle compositions and structure with hydrophobic anti-atherosclerotic agents.

FIGS. 2A through 2D illustrate an exemplary embodiment: 2A) a mixture of phosphatidylcholine, cholesterol, chitosan, and EGCG in 1×PBS; 2B) chitosan coated EGCG encapsulated liposomes (CSLIPO-EGCG) in 1×PBS; 2C) scanning electron microscope image of CSLIPO-EGCG; and 2D) the size of CSLIPO-EGCG measured using Brookhaven BI-MAS particle size analyzer.

FIGS. 3A and 3B illustrate results of exemplary stability analysis, showing stability of 0.5 mM of native EGCG and equivalent amounts of EGCG encapsulated into liposomes (LIPO) and CSLIPO in 1×PBS (pH 7.2) at 4° C. (3A) and 25° C. (3B). Means at a time without a common superscript differ, P<0.05.

FIGS. 4A though 4C illustrate results of exemplary analysis: fluorescent images of cellular uptake of 1×PBS as control (4A), NBD-labeled LIPO (4B), and NBD-labeled CSLIPO (4C) by MCF7 human breast cancer cells. MCF7 cells were incubated with the above treatments for 1 hour, 2 hours and 4 hours at 37° C. Cell nuclei were stained blue by DAPI (λₑₓ=358 nm, λₑₘ=461 nm) and merged with green fluorescent signals from NBD-labeled liposomes (λₑₓ=460 nm, λₑₘ=535 nm).

FIGS. 5A and 5B illustrate results of exemplary analysis: EGCG content in MCF7 cells after treating them with 50 μM (5A) and 100 μM (5B) of LIPO-EGCG and CSLIPO-EGCG for 4 hours at 37° C. Values are the means of four independent experiments, with standard deviations represented by vertical bars. Bars without a common superscript differ, P<0.01.

FIG. 6 illustrates results of exemplary analysis: a transmission electron microscope (TEM) image of lipid nanoparticles stained with 2% of uranyl acetate.

FIGS. 7A through 7D illustrate results of exemplary analysis: stability of nanoencapsulated and native EGCG (nanostructured lipid carriers, NLC; chitosan coated NLC, CSNLC) in 1×PBS at pH 1, 3, 5 and 7.4 in FIGS. 7A, 7B, 7C and 7D, respectively (n=3).

FIGS. 8A through 8C illustrate results of exemplary analysis: stability of nanoencapsulated and native EGCG (NLC and CSNLC) in 1×PBS at pH 7.4 at 4, 22 and 37° C. in FIGS. 8A, 8B and 8C respectively (n=3).

FIG. 9 illustrates results of exemplary analysis: Stability of nanoencapsulated and native EGCG (NLC and CSNLC) in RPMI1640 medium at 37° C., (A) RPMI1640 medium without SOD; (B) RPMI1640 medium with SOD 5 U/ml (n=3).

FIGS. 10A through 10D illustrate results of exemplary analysis: Stability of nanoencapsulated and native EGCG (NLC and CSNLC) in RPMI1640 medium at 4 or 37° C. and with or without SOD incubated with THP-1 macrophage cells. (10A) cell RPMI1640 medium at 4° C. without SOD; (10B) cell RPMI1640 medium at 4° C. with SOD 5 U/ml; (10C) cell RPMI1640 medium at 37° C. without SOD; (10D) cell RPMI1640 medium at 37° C. with SOD 5 U/ml (n=3).

FIG. 11 illustrates results of exemplary analysis: Cellular EGCG content in THP-1 derived macrophages treated by 100 μM of native EGCG, and EGCG encapsulated NLC (NLCE) and EGCG encapsulated CSNLC (CSNLCE) in the complete medium including SOD (5 U/ml) at 4° C. or 37° C. and at 2 h or 4 h of incubation. Compared with native EGCG, CS increase EGCG content *: p<0.05; * *: p<0.01. Compared with NLCE, CSNLCE increase EGCG content a: p<0.01 (n=3).

FIG. 12 illustrates results of exemplary analysis: Viability of THP-1 derived macrophages treated by 5, 10, and 20 μM of VNLC, VCSNLC, NLCE, CSNLCE, EGCG and 1×PBS. Data are means±SD (n=3).

FIGS. 13A and 13B illustrate results of exemplary analysis: Effects of NLCE, CSNLCE, VNLC, VCSNLC, native EGCG and 1×PBS on cholesterol levels of macrophages differentiated from THP-1 cells. (13A), The macrophages were treated with the above six treatments without oxLDL for 18 h; (13B), The macrophages were starved for 8 h first and then incubated with 40 mg protein/ml of minimally modified-LDL (oxLDL) and the same six treatments the above mentioned for 18 h. Data are mean±SD, n=3. Symbol * in figure panels indicates a significant difference from control 1*PBS, EGCG, VNLC and VCSNLC with P<0.05; Symbol ** indicates P<0.01.

FIGS. 14A and 14B illustrate results of exemplary analysis: Fluorescent microscopy images of NBD-labeled NLC and NBD-labeled CSNLC uptake by cells after 2-, 4-, 6-, 18-, 24-hour incubation at 37 degree temperature. (14A) NLC; (14B) CSNLC Upper panels: NBD-labeled nanoparticles (Green); Lower panels:DAPI-stained nuclei (Blue). All images are 10× optical magnification.

FIGS. 15A through 15C illustrate results of exemplary analysis: Binding assay of liposomes to THP-1 derived macrophages. (15A) Ligand-liposome (composed of 30 mol % KDdiA-PC); (15B) Control-liposome (no KDdiA-PC); (15C) 1×PBS. Liposomes were labeled with 7-Nitro-2-1,3-benzoxadiazol-4-yl (NBD)-PE (1.0 mol % relative to the total lipid) as fluorescence dye (λ of excitation is 460 nm, λ of emission is =535 nm) (green color). Cell nuclei were stained by DAPI (λ of excitation is 358 nm, λ of emission is 461 nm) (blue color).

FIGS. 16A through 16D illustrate results of exemplary analysis: KDdiA-PC-liposomes target to atherosclerosis in LDL receptor null mice (a well-known atherosclerosis animal model). The KDdiA-PC containing liposome vesicle and control liposome vesicles, which were labeled with 1,1′-dioctadecyl-3,3,3′,3′-tetramethyl indotricarbocyanine iodide (DiR) near infrared (NIR)fluorescence dye (λ of excitation is 730 nm, λ of emission is 790 nm), were intravenously injected through tail vein. Twenty hours later, NIR images combined with X-ray images were obtained from the left side (16A) and right side (16B), or after exposing the aorta by cutting the abdomen open (16C) and isolated aorta from each mouse (16D) using an IVIS® Lumina XR imaging system. Mouse on the left side was injected with liposomes containing KDdiA-PC, the mouse on the right side was injected with control liposomes without KDdiA-PC.

FIG. 17 illustrates results of exemplary analysis: KOdiA-PC increased the binding affinity of liposomes to macrophages. Human monocytic THP-1 cells were cultured in RPMI 1640 medium supplemented with 10% fetal bovine serum and 0.05 mM of 2-mercaptoethanol. The cells were incubated at 37° C., 95% humidity, and an atmosphere of 5%. Cells were differentiated into macrophages by incubating them with 50 ng/ml PMA for 72 hours. Macrophages derived from THP-1 cells were treated with 7-nitro-2-1,3-benzoxadiazol-4-yl (NBD)-PC-labeled liposomes with target ligand KOdiA-PC or NBD-labeled liposomes without KOdiA-PC for 2 hour at 4° C. NBD-labeled liposomes were green (λ of excitation is 460 nm, λ of emission is =535 nm). Cell nuclei were stained by DAPI (λ of excitation is 358 nm, λ of emission is 461 nm) (blue color). Liposomes with KOdiA-PC had significantly higher binding affinity to macrophages and increased uptake of liposomes by macrophages compared to liposomes without KOdiA-PC.

FIGS. 18A-18D illustrate results of exemplary analysis: macrophages derived from THP-1 cells were treated with NBD-labeled liposomes with or without target ligands (KOdiA-PC) and subject to staining by ligand, DAPI, and CD36. 18A) shows the comparative staining results from cells treated with liposomes without KOdiA-PC; 18B) shows the comparative staining results from cells treated with liposomes with KOdiA-PC; 18C) shows the comparative staining results from cells treated with liposomes without KOdiA-PC in the presence of anti-CD36; and 18D) shows the comparative staining results from cells treated with liposomes with KOdiA-PC in the presence of anti-CD36.

FIGS. 19A and 19B illustrate results of exemplary analysis: 19A) the presence of CD36 and DAPI staining are compared in control macrophages and CD36 knockdown macrophages; and 19B) binding assay of NBD-labelled liposomes with KOdiA-PC by control macrophages and CD36 knockdown macrophages.

FIGS. 20A-20F illustrate results of exemplary analysis: 20A-20C show that ligand didn't increase the cell binding affinity of liposomes to 3T3-L1 preadipocytes (cells with low expression level of CD36). 20A) 3T3-L1 cells treated with liposomes without ligands; 20B) 3T3-L1 cells treated with liposomes carrying KOdiA-PC; and 20C) expression of CD36 in 3T3-L1 preadipocytes indicated by anti-CD36 antibodies. 20D-20F show that KOdiA-PC increases the binding affinity of liposomes to 3T3-L1 adipocytes (cells with high expression level of CD36). 20D) 3T3-L1 adipocytes treated with untargeted vesicle; 20E) 3T3-L1 adipocytes treated with targeted vesicle; and 20F) CD36 expression in 3T3-L1 adipocytes indicated by anti-CD36 antibodies.

FIG. 21 illustrate results of exemplary analysis, showing the size and distribution of NLC-EGCG as measured by means of Brookhaven BI-90 particle size analyzer. The particle size is about 60 nm in diameter.

FIG. 22 illustrates results of exemplary analysis, showing that NLC-EGCG with KOdiaA-PC increased cellular EGCG content.

FIG. 23 illustrates results of exemplary analysis, showing that KOdiA-PC increased the binding affinity of NLC-EGCG to macrophages. Macrophages derived from THP-1 cells were treated with NBD-labeled NLC-EGCG with or without KOdiA-PC in PRMI 1640 for 2 hour at 37° C. NLC-EGCG containing KOdiA-PC had significantly higher binding affinity to macrophages and resulted in more uptake of NLC-EGCG by macrophages compared to NLC-EGCG without KOdiA-PC.

FIG. 24 illustrates results of exemplary analysis, showing that KOdiA-PC increased the target specificity of nanocarriers to atherosclerotic lesions in LDL receptor null mice. Male low-density lipoprotein receptor-deficient (LDLr−/−) mice (C57BL6 background) were fed with Harlan Teklad an atherogenic diet (TD.88137) containing 21% of saturated fat (w/w) and 0.15% of cholesterol (w/w) for 24 weeks from 6 weeks old. Mice developed atherosclerotic lesions on aortic arch and abdominal arterial vessels after feeding this atherogenic diet for 24 weeks. Mice were housed at 22 to 24° C., 45% relative humidity and a daily 10/14 light/dark cycle with the light period from 06:00 to 16:00. Food and water were given ad labium. Body weights of mice at the time of experiments were 40-45 g.

FIGS. 25A1-A6 and FIGS. 25B1-B6 illustrate results of exemplary analysis, showing that nanocarriers containing KOdiA-PC targeted to atherosclerotic lesions (white plaques) in LDL receptor null mice.

FIG. 26 illustrates results of exemplary analysis, showing target specificity of nanocarriers to atherosclerotic lesions in LDLr−/− mice. These are representative images from the cross-sections of aortic arches, where Cy7 is shown in blue, auto-fluorescence in green and oil in red denotes atherosclerotic lesions (plaques).

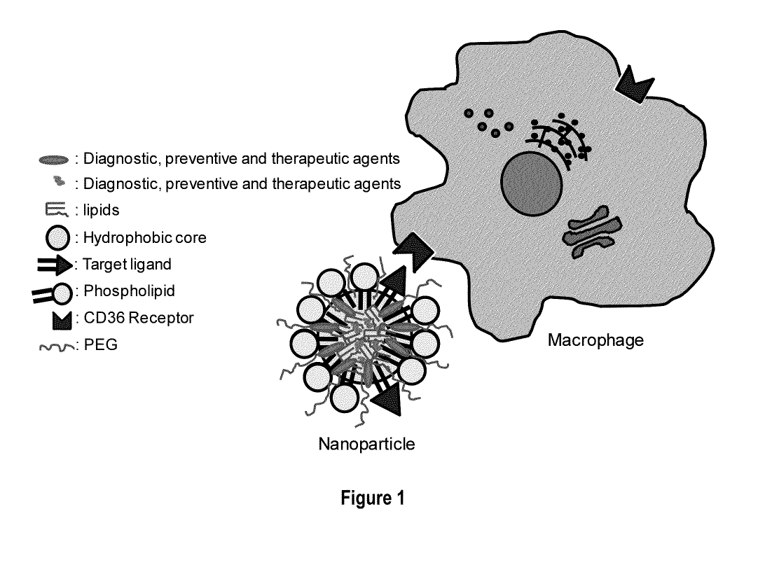

### DETAILED DESCRIPTION OF THE INVENTION

### Definitions

Unless otherwise noted, terms are to be understood according to conventional usage by those of ordinary skill in the relevant art.

As used herein, the term “nanoparticles,” “nanocarriers,” or “nanoliposomes” encompasses but is not limited to, liposomes, polymersomes, microspheres, and nano- or micro-structured lipid carriers, high-density lipoprotein particles. Nanoparticles can be manufactured by any method known in the art, including but not limited to conventional mixing, dragee-making, sputtering, emulsifying, sonicating, entrapping, encapsulating, lyophilizing, and phase inverse-based processes. In some embodiments, the nanoparticles comprise oxidized phospholipids for targeting atherosclerotic lesion.

As the term “bioactive agent” encompasses any diagnostic, therapeutic, preventive and nutrient molecules that can be included in the nanoparticles comprising oxidized phospholipids. In some embodiments, the terms “bioactive agent” and “diagnostic and anti-atherosclerotic agents” are used interchangeably and encompass any agent that can be used for diagnosis, prevention and treatment of atherosclerosis. For example, anti-atherosclerotic agents include but are not limited to, cholesterol medications, anti-platelet medications, beta blocker medications, angiotensin-converting enzyme (ACE) inhibitors, water pills (diuretics) and medications for controlling specific risk factors for atherosclerosis, such as diabetes. Diagnostic tests include but are not limited to blood tests, electrocardiograms, chest x-ray, ankle/brachial index, echocardiography, ultrasound, computed tomography (CT) scan, positron emission tomography and computed tomography (PET/CT), magnetic resonance imaging (MRI), stress test, or angiograph. In some embodiments, a diagnostic agent for atherosclerosis includes a fluorescent dye. In some embodiments, anti-atherosclerotic agents include catechins.

As used herein, the term “oxidized phospholipids” encompasses an oxidized form of any phospholipids. Representative examples of known synthetic phospholipids include, without limitation, 1-palmitoyl-2-azelaoyl-sn-glycero-3-phosphocholine, 1-hexadecyl-2-azelaoyl-sn-glycero-3-phosphocholine, 1-palmitoyl-2-glutaroyl-sn-glycero-3-phosphocholine (PGPC), 1-palmitoyl-2-(5-oxovaleroyl)-sn-glycero-3-phosphocholine (POVPC), and 1-palmitoyl-2-(9-oxononanoyl)-sn-glycero-3-phosphocholine. In some embodiments, the oxidized phospholipids comprise KDdiA-PC, HOdiA-PC. In some embodiments, the oxidized phospholipids comprise KOdiA-PC. In some embodiments, the oxidized phospholipids comprise a mixture of KDdiA-PC and KOdiA-PC.

As used herein, the term “scavenger receptor” encompasses any receptor recognize modified low-density lipoprotein (LDL) by oxidation or acetylation. They are separated into classes A, B, and C, including but not limited to, for example, those located on intimal macrophages. Exemplary scavenger receptors include but are not limited to scavenger receptor A (the first OxLDL receptor to be characterized and cloned, CD36, CD68, Lectin-like oxidized LDL receptor-1 (LOX-1), SR-A1, SR-B1 and etc.

### Atherosclerotic Cardiovascular Disease and Oxidized Phospholipids

Atherosclerotic cardiovascular disease is the No. 1 killer in the United States and other developed countries. Since the disease cannot be detected at early stage, it is also a silent killer. There is a critical need to develop a method or technique to detect it at early stage, and prevent and treat it using a target delivery method.

Atherosclerotic vascular disease arises as a consequence of the deposition and retention of serum lipoproteins in the artery wall. Macrophages in lesions have been shown to express ≧6 structurally different scavenger receptors for uptake of modified forms of low-density lipoproteins (LDLs) that promote the cellular accumulation of cholesterol. Because cholesterol-laden macrophage foam cells are the primary component of the fatty streak, the earliest atherosclerotic lesion, lipid uptake by these pathways has long been considered a requisite and initiating event in the pathogenesis of atherosclerosis. Scavenger receptors are known to play important roles in sterile inflammation and infection. Their regulation and signal transduction and the potential impact of these pathways in regulating the balance of lipid accumulation and inflammation in the artery wall are important for diagnostic and therapeutic purposes.

Macrophages play an important role in the development of atherosclerosis. After taking up choelsterol-riched oxLDL, more cholesterol will be accumulated in the macrophages, which are called foam cells. After foam cells are dead, the cholesterol and other lipid deposit on the arterial wall and form a plaque. Those foam cells can also release many inflammatory factors to amplify local inflammatory response and recruit more macrophages into the arterial wall, which can result in more foam cells and further more and larger atherosclerotic lesions. Macrophages take up oxLDL using scavenger receptors. Macrophage scavenger receptor CD36 is a major membrane protein involved in the uptake of cholesterol-rich modified lipoproteins, such as oxLDL. CD36 correlates well with lesion severity.

Oxidized phospholipids have high binding affinities to the oxLDL binding sites of CD36 and participate in CD36-mediated recognition and uptake of particles by intimal macrophages. Therefore, oxidized phospholipids can increase nanoparticle target specificity. Oxidized phospholipids include HDdiA-PC and HOdiA-PC, the 9-hydroxy-10-dodecenedioic acid and 5-hydroxy-8-oxo-6-octenedioic acid esters of 2-lyso-PC; HODA-PC and HOOA-PC, the 9-hydroxy-12-oxo-10-dodecenoic acid and 5-hydroxy-8-oxo-6-octenoic acid esters of 2-lyso-PC; KODA-PC and KOOA-PC, the 9-keto-12-oxo-10-dodecenoic acid and 5-keto-8-oxo-6-octenoic acid esters of 2-lyso-PC; KDdiA-PC and KOdiA-PC, the 9-keto-10-dodecendioic acid and 5-keto-6-octendioic acid esters of 2-lyso-PC; OV-PC and ON-PC, the 5-oxovaleric acid and 9-oxononanoic acid esters of 2-lyso-PC; POVPC, 1-palmitoyl-2-(5-oxovaleroyl)-phosphatidylcholine, and etc. KDdiA-PC and KOdiA-PC have the high binding affinity to macrophage CD36.

Some non-limiting examples of atherosclerotic cardiovascular diseases are coronary heart disease, myocardial infarction, acute coronary syndromes, angina pectoris, myocardial ischemia, stroke, cerebrovascular inflammation, cerebral hemorrhage.

### Compositions for Diagnosis, Prevention and Treatment of Atherosclerosis

In one aspect, provided herein are nanoparticles comprising one or more oxidized phospholipids that are encapsulated within, adhered to a surface of, or integrated into the structure of the nanoparticles. The oxidized phospholipids target an atherosclerotic lesion site, by binding to scavenger receptor on the macrophages.

In one aspect, nanoparticles coated with oxidized phospholipids and variants thereof (e.g., KDdiA-PC) are used for targeted delivery of diagnostic, preventive, therapeutic, and/or nutrients to the atherosclerotic lesion for diagnosis, prevention and treatment of atherosclerosis.

In some embodiments, the oxidized phospholipids and variants thereof themselves form the nanoparticles.

Nanoparticulate technology offers advantages in the diagnosis and treatment of atherosclerotic cardiovascular disease because most biological processes, including atherosclerosis, occur at the nanoscale. Nanoparticle-based delivery system has been used to protect and deliver poorly soluble drugs effectively. Smaller nanoparticles are extravagated effectively into tissues and prolong the circulation time. The nanoparticles can have different shapes and compositions. But they must have phospholipid surface, or similar structure, which can allow oxidized phospholipids to incorporate into the surface phospholipid layer of nanoparticles. Both hydrophilic and hydrophobic therapeutic compounds can be encapsulated or incorporated into nanoparticles. Nanoparticles can increase diagnostic and anti-atherosclerotic compounds absorption, protect compounds from premature degradation, prolong compounds circulation time, exhibit high differential uptake efficiency in the target cells (or tissue) over normal cells (or tissue), lower toxicity through preventing the compounds from prematurely interacting with the biological environment, improve intracellular penetration, and increase therapeutic effectiveness.

In some embodiments, the nanoparticles comprise liposomes, polymerosomes, microspheres, micro-structured lipid carriers, nano-structured lipid carriers, or a combination thereof.

Any suitable molecules; for example, water soluble polymers, biodegradable polymers, co-polymers, can be used to form the nanoparticles described herein. Any method known in the art can be used to form, encapsulate, incorporate, and/or modify nanoparticles with the oxidized phospholipids described herein.

In some embodiments, one or more molecules forming the nanoparticles comprise albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(-hydroxyvalerate), poly(D,L-lactide-co-glycolide), poly(1-lactide-co-glycolide), poly(-hydroxybutyrate), poly(-hydroxybutyrate), poly(-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, apolioprotein A1, apolioprotein A2, other apolioproteins or a combination thereof.

In some embodiments, one or more molecules forming the nanoparticles comprise biodegradable polymer such as PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride).

Nanoparticles can be of any suitable size or configurations. For example, the nanoparticles have an average linear dimension of between about 5 nanometers to about 5,000 nanometers. In some embodiments, the nanoparticles have an average linear dimension of 10 nanometers or smaller, 20 nanometers or smaller, 30 nanometers or smaller, 40 nanometers or smaller, 50 nanometers or smaller, 60 nanometers or smaller, 70 nanometers or smaller, 80 nanometers or smaller, 90 nanometers or smaller, 100 nanometers or smaller, 110 nanometers or smaller, 120 nanometers or smaller, 130 nanometers or smaller, 140 nanometers or smaller, 150 nanometers or smaller, 160 nanometers or smaller, 170 nanometers or smaller, 180 nanometers or smaller, 200 nanometers or smaller, 210 nanometers or smaller, 220 nanometers or smaller, 230 nanometers or smaller, 240 nanometers or smaller, 250 nanometers or smaller, 300 nanometers or smaller, 350 nanometers or smaller, 400 nanometers or smaller, 450 nanometers or smaller, 500 nanometers or smaller, 550 nanometers or smaller, 600 nanometers or smaller, 650 nanometers or smaller, 700 nanometers or smaller, 800 nanometers or smaller, 900 nanometers or smaller, 1,000 nanometers or smaller, 1,100 nanometers or smaller, 1,200 nanometers or smaller, 1,400 nanometers or smaller, 1,600 nanometers or smaller, 1,800 nanometers or smaller, 2,000 nanometers or smaller, 2,500 nanometers or smaller, 3,000 nanometers or smaller, 3,500 nanometers or smaller, 4,000 nanometers or smaller, or 5,000 nanometers or smaller.

In some embodiments, a larger nanoparticle contains within itself one or more smaller nanoparticles. For example, the small nanoparticles are formed by one or more bioactive agents (e.g., a drug). Alternative, the one or more bioactive agents are encapsulated within, adhered to a surface of, or integrated into the structure of the small nanoparticles.

In one aspect, the nanoparticles comprising oxidized phospholipids further comprise one or more additional targeting ligands that also target an atherosclerotic lesion site. Such additional targeting ligands include but are not limited to, for example, antibodies to oxidized LDL, scavenger receptor A (the first OxLDL receptor to be characterized and cloned, CD36, CD68, Lectin-like oxidized LDL receptor-1 (LOX-1), SR-A1, SR-B1, or a combination thereof.

In one aspect, the nanoparticles comprising oxidized phospholipids are multifunctional nanodelivery systems that can simultaneously encapsulate and/or incorporate diagnostic, preventive and therapeutic agents in one nanoparticle delivery system or more nanoparticle delivery systems for more efficient prevention, diagnosis and treatment of atherosclerosis.

In some embodiments, the nanoparticles comprise a labeling agent, alone or in combination with any other active agents. The labeling agent can be any reagent that can provide an indicium to suggest the presence of an atherosclerotic lesion site. In some embodiments, the indicium is a light signal; e.g., a light at a certain wavelength, a light that emits a specific color. Such labeling agents include but are not limited to a fluorescent dye, a fluorescent protein (e.g., green, yellow or red fluorescent protein), a radioactive label, a biomarker, a reagent that binds to a biomarker near or at an atherosclerotic lesion site, an antibody, a secondary antibody, and an imaging reagent.

In some embodiments, an imaging reagent can be delivered to an atherosclerotic lesion site in a patient or animal using the nanoparticles before the patient or animal is subject to live imaging analysis.

In some embodiments, oxidized phospholipids provided are conjugated directly with the additional targeting ligand and/or the labeling agent; see, for example, fluorescent oxidized phospholipids as disclosed herein.

Nanoparticles offer many advantages when used in delivering bioactive agents. Nanoparticles can increase target specificity of existing diagnostic and anti-atherosclerotic agents. Many diagnostic and anti-atherosclerotic agents have a low level of target specificity. Normal tissues have a normal and intact vasculature; however, disease tissues have a leaky vasculature. The small size of nanoparticles allows them to enter the disease tissues, such as atherosclerotic lesions. Incorporation a targeting ligand (e.g., KDdiA-PC) on the surface of nanoparticles, further dramatically improves the target specificity of nanoparticles to intimal macrophages. Many diagnostic and anti-atherosclerotic agents have a low level of solubility, stability, and bioavailability. Also advantageously, nanoparticles have a hydrophobic core to accommodate more hydrophobic agents to increase their solubility in physiological solution including blood and lymph. After unstable agents are encapsulated into nanoparticles, their stability is also improved. The encapsulated agents are somehow sealed into the nanoparticles, cannot be degraded or metabolized by the exterior enzymes. Further, the nanoparticles are coated with chitosan to enhance their cellular bioavailability. And increased stability, solubility and circulating time can contribute to the increased bioavailability.

In some embodiments, the nanoparticles comprise, all or some of the following components: lipids (such as triglyceride), phospholipid, alpha-tocopherol acetate, polysaccharides (such as chitosan), poly(ethylene glycerol) (PEG), surfactant(s) and cosurfactant(s) (such as polyethylene glycol (15)-hydroxystearate), salt (such as sodium chloride) and water. Diagnostic and anti-atherosclerotic agents, and any other compounds can be encapsulated or incorporated into the nanoparticle.

In some embodiments, multiple levels of encapsulation are possible to prolong the activity of the bioactive agents. For example, a therapeutic agent can be first encapsulated in smaller nanoparticles before they are further encapsulated in larger nanoparticles. Degradation of the outer/larger nanoparticles allows the drug to be released from the inner/smaller nanoparticles; thus allowing the drug to be delivered in an extended period of time. One of skill in the art would understand that it is possible to manipulate the size, thickness and molecular components of the two populations of nanoparticles such that the time length for the extended delivery can be controlled and modified.

In some embodiments, compositions provided herein comprise nanoparticles coated with chitosan, a cellular uptake enhancer, to increase cellular bioavailability; incorporate poly(ethylene glycerol) (PEG) on their surface to maintain their integrity and stability and prolong the circulation of nanocarriers, and use KDdiA-PC as a target ligand to increase target specificity to aortic intimal macrophages.

In one aspect, the nanoparticles comprising oxidized phospholipids are used as targeted nanodelivery systems for the diagnosis of atherosclerosis. Diagnostic dyes/agents are encapsulated or incorporated on/into the nanoparticles for detection of atherosclerosis at any stages, which correlate to the intensity of dyes/agents on the arterial wall.

In one aspect, the nanoparticles comprising oxidized phospholipids are used as targeted nanodelivery systems for the prevention and treatment of atherosclerosis. Preventive and therapeutic agents are encapsulated or incorporated on/into the nanoparticles. Their stability, solubility, bioavailability, target specificity and functional efficiency are improved. They can prevent cholesterol accumulation in macrophages and inhibit foam cells formation.

In one aspect, the nanoparticles comprising oxidized phospholipids are used as targeted nanodelivery systems for the prevention and treatment of an inflammatory disorder, an autoimmune disease, and other immune mediated diseases.

In one aspect, the nanoparticles comprising oxidized phospholipids can be used for forensic analysis.

### Delivery of the Composition

In some embodiments, compositions comprising nanoparticles coated with oxidized phospholipids (e.g., KDdiA-PC) are used as targeting ligands coated on nanoparticle based medicine/nutrient carrier for targeted delivery of diagnostic, therapeutic, and/or nutrients to the atherosclerotic lesion for diagnosis, prevention and treatment of atherosclerosis

In some embodiments, compositions comprising the nanoparticles are delivered to a patient have or suspected having an atherosclerotic lesion via intraarterial or intravenous delivery. In some embodiments, intraarterial or intravenous delivery comprises using a catheter. In some embodiments, intraarterial delivery comprises direct injection.

In some embodiments, the compositions are delivered in a single dose. In some embodiments, the compositions are delivered in multiple doses over an extended period of time.

In some embodiments, the compositions further comprise an adjuvant and/or a pharmaceutically compatible carrier.

### Oxidized Phospholipids

In one aspect, provided herein are oxidized phospholipids and variants thereof are incorporated into nanoparticles. Oxidized phospholipids are enriched in atherosclerotic lesions in animals (Podrez et al., 2002a; Podrez et al., 2002b). They are the major ligands for binding oxLDL to CD36 on intimal macrophages.

In some embodiments, oxidized phospholipids provided herein include an ester of lysophosphatidylcholine such as an ester of 1-lysophosphatidylcholine (1-lysoPC) or an ester of 2-lysophosphatidylcholine (2-lysoPC).

In some embodiments, oxidized phospholipids provided herein include 9-hydroxy-10-dodecenedioic acid esters of 2-lyso-PC (HDdiA-PC), 5-hydroxy-8-oxo-6-octenedioic acid esters of 2-lyso-PC(HOdiA-PC), 9-hydroxy-12-oxo-10-dodecenoic acid esters of 2-lyso-PC(HODA-PC), 5-hydroxy-8-oxo-6-octenoic acid esters of 2-lyso-PC(HOOA-PC), 9-keto-12-oxo-10-dodecenoic acid esters of 2-lyso-PC (KODA-PC), 5-keto-8-oxo-6-octenoic acid esters of 2-lyso-PC (KOOA-PC), 9-keto-10-dodecendioic acid esters of 2-lyso-PC (KDdiA-PC), and 5-keto-6-octendioic acid esters of 2-lyso-PC (KOdiA-PC), 5-oxovaleric acid esters of 2-lyso-PC (OV-PC), and 9-oxononanoic acid esters of 2-lyso-PC (ON-PC), 1-palmitoyl-2-(5-oxovaleroyl)-phosphatidylcholine (POVPC), and etc.

In some embodiments, oxidized phospholipids provided herein include but are not limited to 1-palmitoyl-2-azelaoyl-sn-glycero-3-phosphocholine, 1-hexadecyl-2-azelaoyl-sn-glycero-3-phosphocholine, 1-palmitoyl-2-glutaroyl-sn-glycero-3-phosphocholine (PGPC), 1-palmitoyl-2-(5-oxovaleroyl)-sn-glycero-3-phosphocholine, 1-palmitoyl-2-(9-oxononanoyl)-sn-glycero-3-phosphocholine, 1-hexadecyl-2-acetoyl-sn-glycero-3-phosphocholine, 1-octadecyl-2-acetoyl-sn-glycero-3-phosphocholine, 1-hexadecyl-2-butyroyl-sn-glycero-3-phosphocholine, 1-octadecyl-2-butyroyl-sn-glycero-3-phosphocholine, 1-palmitoyl-2-acetoyl-sn-glycero-3-phosphocholine, 1-octadecenyl-2-acetoyl-sn-glycero-3-phosphocholine, 1-hexadecyl-2-(homogammalinolenoyl)-sn-glycero-3-phosphocholine, 1-hexadecyl-2-arachidonoyl-sn-glycero-3-phosphocholine, 1-hexadecyl-2-eicosapentaenoyl-sn-glycero-3-phosphocholine, 1-hexadecyl-2-docosahexaenoyl-sn-glycero-3-phosphocholine, 1-octadecyl-2-methyl-sn-glycero-3-phosphocholine, 1-hexadecyl-2-butenoyl-sn-glycero-3-phosphocholine, Lyso PAF C16, Lyso PAF C18, 1-O-1′-(Z)-hexadecenyl-2-[12-[(7-nitro-2-1,3-benzoxadiazol-4-yl)-amino]dodecanoyl]-sn-glycero-3-phosphocholine, 1-O-1-(Z)-hexadecenyl-2-oleoyl-sn-glycero-3-phosphocholine, 1-O-1-(Z)-hexadecenyl-2-arachidonoyl-sn-glycero-3-phosphocholine, 1-O-1′-(Z)-hexadecenyl-2-docosahexaenoyl-sn-glycero-3-phosphocholine, 1-O-1-(Z)-hexadecenyl-2-oleoyl-sn-glycero-3-phosphoethanolamine, 1-O-1′-(Z)-hexadecenyl-2-arachidonoyl-sn-glycero-3-phosphoethanolamine, and 1-O-1′-(Z)-hexadecenyl-2-docosahexaenoyl-sn-glycero-3-phosphoethanolamine. One of skill in the art would understand that phosphocholine is an intermediate in the synthesis of phosphatidylcholine. In some embodiments, the terms phosphocholine and phosphatidylcholine are used interchangeably.

In some embodiments, truncated versions of oxidized phospholipids are used. Truncated versions of oxidized phospholipids can also have the same high binding affinity to macrophage CD36 receptors as the intact oxidized phospholipids. KDdiA-PC has been isolated and identified from oxLDL (Boullier et al., 2001; Boullier et al., 2000; Podrez et al., 2002a; Podrez et al., 2002b; Watson et al., 1997). KDdiA-PC confers CD36 binding affinity more potently than any hydroperoxy phospholipid species, and may be one of the more important structural and functional determinants of oxLDL (Berliner et al., 1997; Boullier et al., 2000; Leitinger et al., 1999; Watson et al., 1997).

In some embodiments, oxidized phospholipids provided herein include fluorescent oxidized phospholipids. In some embodiments, the oxidized phospholipids include 1-palmitoyl-2-glutaroyl-sn-glycero-3-phospho-N-(3-[4,4-difluoro-4-bora-3a-,4a-diaza-s-indacene]-propionyl)-ethanolamine (BODIPY-PGPE),1-palmitoyl-2-(5-oxovaleroyl)-sn-glycero-3-phospho-N-(3-[4,4-difluoro-4-b-ora-3a,4a-diaza-s-indacene]-propionyl)-ethanolamine (BODIPY-POVPE),1-palmitoyl-2-oleoyl-sn-glycero-3-phospho-N-(3-[4,4-difluoro-4-bora-3a,4a- -diaza-s-indacene]-propionyl)-ethanolamine (BODIPY-POPE), or a combination thereof.

Additional examples of oxidized phospholipids can be found in, for example, U.S. Pat. No. 7,973,023 to Harats et al.; U.S. Pat. No. 7,906,674 to Hermetter et al., each of which is hereby incorporated by reference herein in its entirety.

### Synthesis of Oxidized Phospholipids

In one aspect, provided herein are methods for synthesizing the oxidized phospholipids and variants thereof. The oxidized phospholipids and variants are incorporated into nanoparticles.

Previously, KDdiA-PC was synthesized from 2-Lyso-PC and 8-(2-furyl)octanoic acid, which could be readily synthesized from 1,8-octanediol in 5 steps. After condensation and two stages' oxidation, KDdiA-PC was obtained in pure form after simple silica gel chromatograph purification (Sun et al, 2003).

In some embodiments, the oxidized phospholipids and variants thereof (e.g., KDdiA-PC) are synthesized according to literature with slightly improvements. For example, after 8 steps from 1,8-octanediol, KDdiA-PC was obtained in pure form with preparative TLC purification. All the analytical data were consistent with the literature reporting (Example 1).

Since numerous anti-atherosclerotic agents and diagnostic agents have a low level of bioavailability and target specificity, there is a critical need for engineered carriers to enhance their cellular bioavailability and target specificity for disease prevention, diagnosis and treatment. Nanoparticle based technology is very promising for diagnosis, prevention and treatment of atherosclerosis. Nanoparticles can carry both hydrophilic and hydrophobic compounds, and numerous studies have shown that nanoparticles can increase bioavailability, solubility, stability and payload of diagnostic, preventive and therapeutic compounds, lower their toxicity, prolong their circulation time. They represent a new delivery method for poorly soluble compounds.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Synthesis of KDdiA-PC

An exemplary overall reaction scheme for synthesizing KDdiA-PC is shown below.

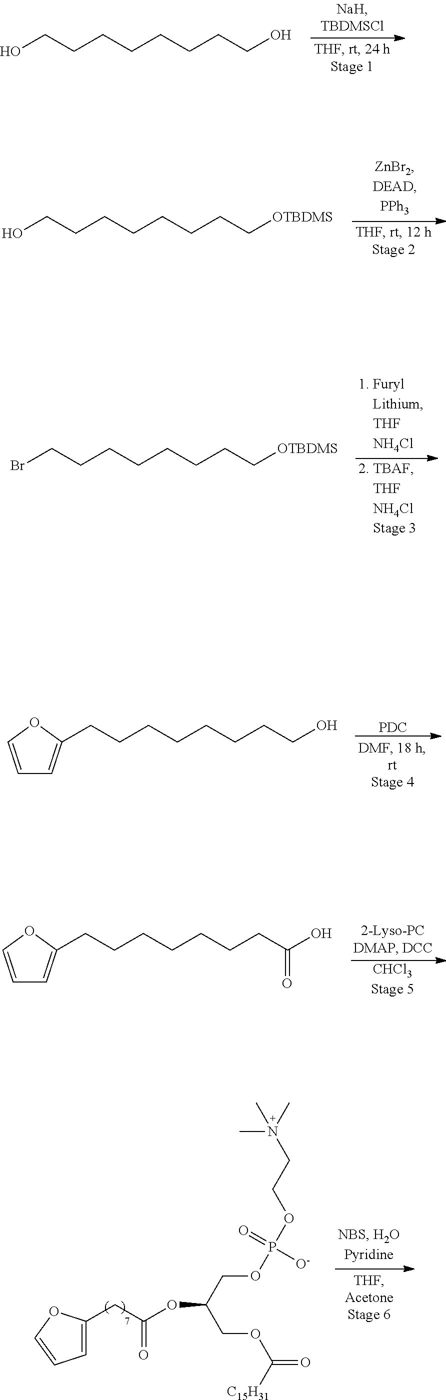

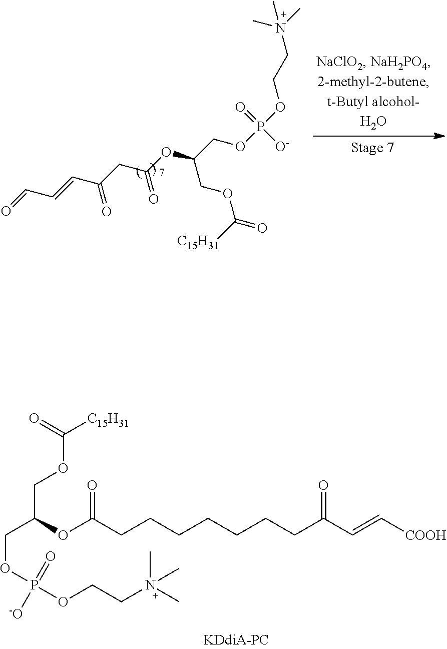

### Description of the Synthetic Process

8-(1,1,2,2-Tetramethyl-1-silapropoxy)octan-1-ol is synthesized as follows:

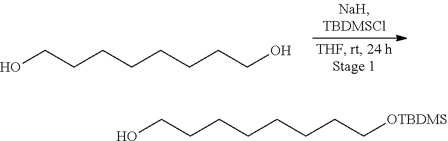 [SMILES: OCCCCCCCCO ; CC(C)(C)[Si](C)(C)OCCCCCCCCO]

Sodium hydride (900 mg, 37.5 mmol) washed with hexanes and suspended in THF (50 mL) 1,8-Octanediol (5 g, 34.2 mmol.) was added to the suspension and stirred for 24 h. (TBDMS)-Cl (5.2 g, 34.5 mmol) was added and stirred for 4 h. The crude reaction mixture was filtered and the solvent was removed on rotary evaporator. Flash chromatography of the crude reaction mixture using ethyl acetate and hexanes afforded the title monosilyl ether in very low yield (1.5 g, 17%). TLC (ethyl acetate/hexanes, 3:17, R_{f}=0.3) stained in KMnO₄ stain. ¹H NMR (400 MHz, CDCl₃) δ 3.57 (q, J=6.8 Hz, 4H), 1.92 (s, 1H), 1.56-1.44 (m, 4H), 1.27 (s, 8H), 0.86 (s, 9H), 0.01 (s, 6H); ¹³C NMR (100 MHz, CDCl₃) δ3.26, 62.83, 32.77, 32.69, 29.36, 29.34, 25.92, 25.67, 25.65, 25.61, 18.31, −5.32.

8-Bromo-1-(1,1,2,2-tetramethyl-1-silapropoxy)octane is synthesized as follows:

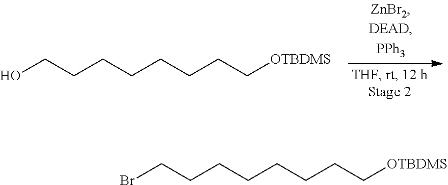 [SMILES: CC(C)(C)[Si](C)(C)OCCCCCCCCO ; CC(C)(C)[Si](C)(C)OCCCCCCCCBr]

To the stirred solution of 8-(1,1,2,2-Tetramethyl-1-silapropoxy)octan-1-ol (1.58 g, 6.06 mmol) and Ph₃P (4.77 g, 18.19 mmol) in THF (76 mL) under Argon, ZnBr₂ (1.36 g, 6.06 mmol) dissolved in THF (63 mL) was added dropwise slowly ZnBr₂ is very hygroscopic and it should be weighed in dry and preferably inert atmosphere. After stirring the reaction mixture for 10 min, diethyl azodicarboxylate (DEAD) (4.22 g, 24.26 mmol) dissolved in THF (25 mL) was added slowly dropwise and the resulting mixture was stirred overnight. The reaction should be done in the absence of light as DEAD is light sensitive. It would be better if the round bottomed flask is covered with an aluminum foil. The precipitate was filtered and the solvent was removed under reduced pressure. Flash chromatography of the crude reaction mixture using ethyl acetate and hexanes afforded the title bromide (819 mg, 63%). TLC (ethyl acetate/hexanes, 1:24, R_{f}=0.30) stained in KMnO₄ stain. ¹H NMR (400 MHz, CDCl₃) δ 3.56 (t, J=6.4 Hz, 2H), 3.36 (t, J=6.8 Hz, 2H), 1.82 (p, J=6.8 Hz, 2H), 1.52-1.36 (m, 4H), 1.28 (s, 6H), 0.87 (s, 9H), 0.01 (s, 6H); ¹³C NMR (100 MHz, CDCl₃) δ 63.26, 33.90, 32.88, 29.30, 28.83, 28.19, 26.04, 25.78, 18.41, −5.19.

8-(2-Furyl)octan-1-ol is synthesized as follows:

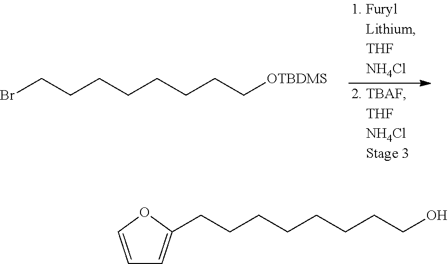 [SMILES: CC(C)(C)[Si](C)(C)OCCCCCCCCBr ; OCCCCCCCCc1ccco1]

A solution of furan (1.39 mL, 19.14 mmol) in dry THF (45 mL) was stirred under argon and cooled in ethylene glycol-dry ice bath (−15° C.) and n-butyllithium (2.41 M, 7.22 mL, 17.41 mmol) was added slowly by means of syringe pump (1.5 mL/min). After complete addition, the solution was stirred for another 30 min at −15° C. and the ethylene glycol-dry ice bath was replaced with an ice bath and the solution was stirred for 1.5 h at 0° C. to generate furyl lithium. 8-Bromo-1-(1,1,2,2-tetramethyl-1-silapropoxy)octane (619 mg, 1.91 mmol) dissolved in 2.5 mL of THF was added to the solution. The solution was stirred at 0° C. for 1 h and then warmed to room temperature. After 7 h the reaction was quenched with saturated NH₄Cl (10 mL) and the mixture was extracted with hexane. The organic layer was dried using anhydrous sodium sulfate, filtered and concentrated under reduced pressure. Without purifying the crude mixture, THF (6 mL) and TBAF (1M in THF, 7.65 mL, 7.65 mmol) were added sequentially and stirred at room temperature. After 5 h, the reaction was quenched with saturated NH₄Cl (12 mL) and the resulting mixture was extracted with ethyl acetate. The combined organic layers were dried with anhydrous sodium sulfate, filtered and concentrated under reduced pressure. Flash chromatography of the crude product using ethyl acetate and hexanes afforded furyl octanol (256 mg, 68%). TLC (25% ethyl acetate/hexanes, R_{f}=0.24) stained in KmNO₄ stain. ¹H NMR (400 MHz, CDCl₃) δ 7.27 (s, 1H), 6.25 (s, 1H), 5.94 (s, 1H), 3.60 (t, J=5.4 Hz, 2H), 2.59 (t, J=7.3 Hz, 2H), 1.76 (s, 1H), 1.68-1.44 (m, 4H), 1.31 (s, 8H); ¹³C NMR (100 MHz, CDCl₃) δ 156.55, 140.67, 110.08, 104.61, 62.71, 32.74, 29.43, 29.19, 28.09, 28.02, 25.83.

8-(2-Furyl)octanoic Acid is synthesized as follows:

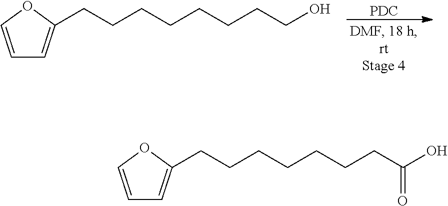

To a solution of 8-(2-Furyl)octan-1-ol (246 mg, 1.25 mmol) in DMF (4 mL) was added PDC (2.83 g, 7.51 mmol) under argon and the resulting reaction mixture was stirred for 18 h at room temperature. The resulting mixture was diluted with saturated NH₄Cl solution (35 mL) and extracted with ethyl acetate. The organic extract was washed with water once and dried over anhydrous sodium sulfate and concentrated under reduced pressure. The excess DMF was removed using high vacuum. The crude product itself is very pure and proceeded as such to the next step (199 mg, 75%). ¹H NMR (400 MHz, CDCl₃) δ 7.27 (s, 1H), 6.25 (s, 1H), 5.95 (s, 1H), 2.60 (t, J=7.3 Hz, 2H), 2.42-2.24 (m, 2H), 1.62 (s, 4H), 1.33 (s, 6H); ¹³C NMR (100 MHz, CDCl₃) δ 180.51, 156.50, 140.74, 110.11, 104.68, 34.22, 29.02, 28.04, 28.00, 24.70.

1-Palmitoyl-2-(8-(2-furyl)octanoyl-sn-glycero-3-phosphatidyl choline is synthesized as follows:

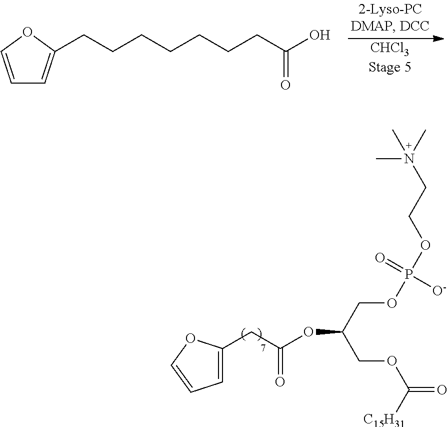

A mixture of furyl octanoic acid (42 mg, 0.2 mmol) and 1-palmitoyl-2-lyso-sn-glycero-3-phosphatidylcholine (50 mg, 0.1 mmol) was dried on high vacuum at room temperature for 6 h and was dissolved in dry CHCl₃ (2 mL, stirred with P₂O₅ over night and distilled). Dicyclohexylcarbodimide (DCC, 120 mg, 1.2 mmol) and N,N-dimethylaminopyridine (12 mg, 0.2 mmol) were sequentially added and stirred for 96 h at room temperature. The crude reaction mixture was concentrated under reduced pressure and purified on preparative TLC using CHCl₃/MeOH/H₂O (16/9/1) to produce 1-Palmitoyl-2-(8-(2-furyl)octanoyl-sn-glycero-3-phosphatidyl choline (40 mg, 58%). ¹H NMR (400 MHz, CDCl₃) δ 7.26 (s, 1H), 6.24 (m, 1H), 5.94 (d, J=3.2 Hz, 1H), 5.17 (m, 1H), 4.43-4.22 (m, 2H), 4.16-4.04 (m, 1H), 4.00-3.85 (m, 2H), 3.79 (s, 3H), 3.34 (s, 9H), 2.58 (t, J=7.7 Hz, 2H), 2.34-2.18 (m, 4H), 1.67-1.47 (m, 6H), 1.39-1.13 (m, 30H), 0.85 (t, J=6.8 Hz, 3H); ¹³C NMR (100 MHz, CDCl₃) δ 173.76, 173.23, 156.47, 140.72, 110.12, 104.67, 70.65, 66.51, 63.50, 63.03, 59.37, 54.57, 34.35, 34.21, 32.00, 29.79, 29.62, 29.45, 29.25, 29.06, 28.06, 28.00, 24.96, 22.77, 14.21.

1-Palmitoyl-2-(9-oxo-12-oxododec-10-enoyl)-sn-glycero-3-phosphatidylcholine (KODA-PC) is synthesized as follows:

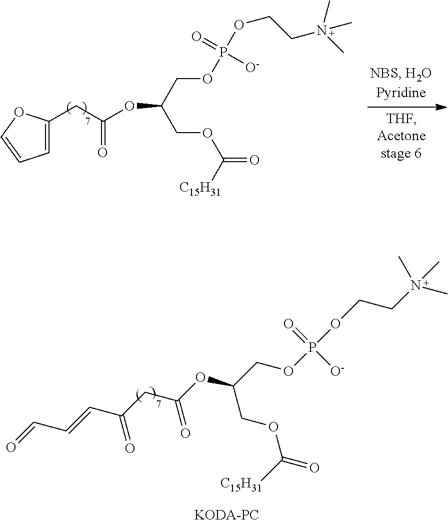

Under argon atmosphere, NBS (4.2 mg, 0.024 mmol) and pyridine (4.4 mg, 0.056 mmol) were sequentially added to a solution of furyl phosphatidylcholine (16 mg, 0.023 mmol) in THF/acetone/water (3 mL, 5/4/2) at −20° C. The resulting mixture was stirred for 1 hour at this temperature and then kept at room temperature for 5 h. The solvent was then removed quickly by rotary evaporation, and the crude KODA-PC mixture was used for next step without further purification. ¹H NMR (CDCl₃, 400 MHz): δ 9.76 (d, J=7.3 Hz, 1H), 6.88 (d, J=15.9 Hz, 1H), 6.75 (dd, J=16.0, 7.4 Hz, 1H), 5.19 (bm, 1H), 4.43 (bs, 2H), 4.31-4.34 (m, 1H), 4.11 (dd, J=11.4, 7.2 Hz, 1H), 3.92-4.07 (m, 4H), 3.41 (bs, 9H), 2.68 (t, J=7.3 Hz, 2H), 2.24-2.31 (m, 4H), 1.54-1.69 (m, 6H), 1.21-1.29 (m, 30H), 0.84 (t, J=6.9 Hz, 3H).

1-Palmitoyl-2-(11-carboxy-9-oxoundec-10-enoyl)-sn-glycero-3-phosphatidylcholine (KDdiA-PC) is synthesized as follows:

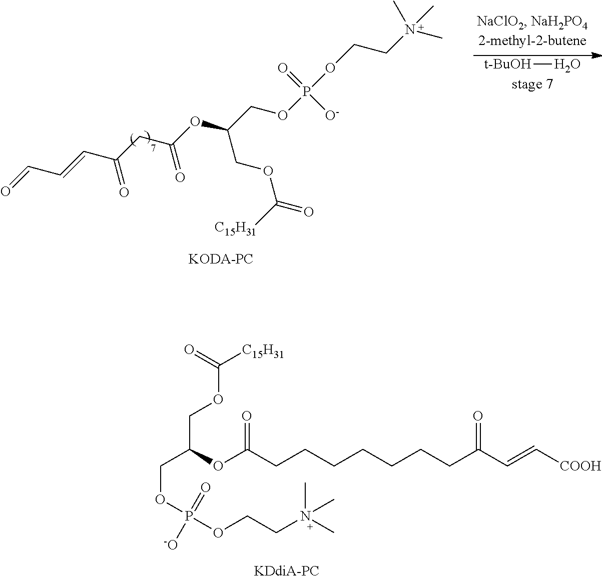

To a magnetically stirred solution of KODA-PC (the crude product of last step, 0.023 mmol) in t-BuOH—H₂O (5:1, v/v, 1.0 mL) was added NaH₂PO₄ (4.6 mg, 0.038 mmol), 2-methyl-2-butene (0.23 mL, 0.46 mmol, 2M solution in THF), and NaClO₂ (0.7 mg, 0.008 mmol, 0.35 eq). The resulting mixture was stirred for 2 h at room temperature under Ar. The solvent was removed. The residue was extracted with 4:1 CHCl₃/MeOH. The crude product was purified by silica gel preparative TLC (CHCl₃/MeOH/H₂O, 11:9:1; R_{f}=0.3) to give KDdiA-PC (8.0 mg, 48% for two steps). ¹H NMR (CD₃OD, 400 MHz): δ 6.67 (d, J=16.0 Hz, 1H), 6.59 (d, J=16.0 Hz, 1H), 5.11 (m, 1H), 4.30 (dd, J=12.4, 3.7 Hz, 1H), 4.16 (m, 2H), 4.06 (dd, J=11.9, 6.9 Hz, 1H), 3.87-3.99 (m, 2H), 3.52-3.54 (m, 2H), 3.11 (s, 9H), 2.55 (t, J=7.4 Hz, 2H), 2.19-2.25 (m, 4H), 1.50 (m, 6H), 1.17-1.23 (30H), 0.78 (t, J=6.9 Hz, 3H).

### Example 2

### Liposomes Improve Stability, Cellular Bioavailability and Function of Green Tea Catechins

The chemopreventive actions exerted by green tea are thought to be due to its major polyphenol, (−)-epigallocatechin-3-gallate (EGCG). However, the low level of stability and bioavailability in the body makes administering EGCG at chemopreventive doses unrealistic. EGCG encapsulated chitosan-coated nanoliposomes (CSLIPO-EGCG) were synthesized, and their antiproliferative and proapoptotic effects in MCF7 breast cancer cells were observed. CSLIPO-EGCG significantly and dramatically enhanced EGCG stability, increased intracellular EGCG content in MCF7 cells, induced apoptosis of MCF7 cells, and inhibited MCF7 cell proliferation compared to native EGCG and void CSLIPO. The CSLIPO-EGCG retained its antiproliferative and proapoptotic effectiveness at 10 μM or lower, at which native EGCG doesn't have any beneficial effects. This study showed that using biocompatible and biodegradable CSLIPO-EGCG can dramatically improve the stability, cellular bioavailability and anti-function of EGCG with minimized immunogenicity and side-effects.

Characteristics of nanoliposomes are summarized in Table 1 and Table 2 as follows.

TABLE 1
Characteristics of nanoliposomes.

| Nano- | Effective | Poly- | Zeta |
|---|---|---|---|
| liposomes | diameter (nm) | dispersity | potential (mV) |
| LIPO-EGCG | 56.0 ± 2.0 | 0.24 ± 0.01 | −6.8 ± 1.8 |
| V.LIPO | 50.6 ± 3.0 | 0.22 ± 0.01 | −9.6 ± 1.9 |
| CSLIPO-EGCG | 85.0 ± 6.6 | 0.35 ± 0.02 | 16.4 ± 2.8 |
| V.CSLIPO | 88.1 ± 8.2 | 0.38 ± 0.01 | 19.2 ± 2.6 |

TABLE 2
Particle size, Zeta potential, and polydispersity of LIPO-EGCG and CSLIPO-
EGCG dissolved in 1 X PBS at pH 7.2 after storage at 4° C. and 25° C.

| Nanoliposomes | | Particle size (nm) | | Zeta potential (mV) | | Polydispersity | |
|---|---|---|---|---|---|---|---|
| Temperature | | 0 day | 7 days | 0 day | 7 days | 0 day | 7 days |
| 4° C. | LIPO-EGCG | 52.0 ± 0.8 | 52.8 ± 1.5 | −7.6 ± 2.9 | −12.1 ± 2.8 | 0.24 ± 0.01 | 0.25 ± 0.02 |
| | CSLIPO-EGCG | 73.0 ± 0.3 | 83.2 ± 2.3 | 18.3 ± 2.4 | 18.6 ± 4.0 | 0.36 ± 0.01 | 0.36 ± 0.01 |
| | | 0 hour | 12 hours | 0 hour | 12 hours | 0 hour | 12 hours |
| 25° C. | LIPO-EGCG | 54.8 ± 0.3 | 61.5 ± 0.5 | −19.1 ± 0.8 | −17.5 ± 2.1 | 0.21 ± 0.01 | 0.25 ± 0.04 |
| | CSLIPO-EGCG | 78.5 ± 0.3 | 80.8 ± 1.1 | 14.3 ± 0.9 | 14.7 ± 1.5 | 0.31 ± 0.01 | 0.37 ± 0.02 |

### Example 3

### Nanostructured Lipid Carriers Improve Stability, Cellular Bioavailability and Function of Green Tea Catechins

Green tea is made from the dried leaves of the Camellia Sinensis plant. Green tea catechins constitute about 33% of total dry tea weight (Wang et al., 2006). EGCG is the most abundant catechin and comprises 48-55% of total catechins (Basu and Lucas, 2007). One 2 g green tea bag contains about 330 mg of EGCG. In vitro studies show that EGCG induce apoptosis of macrophages and macrophage-derived foam cells and inhibit the expression and production of inflammatory factors from those cells (Hashimoto and Sakagami, 2008; Hayakawa et al., 2001; Ichikawa et al., 2004). When apolipoprotein E null mice are treated with daily intraperitoneal injections of EGCG at a dose of 10 mg/kg body weight, cuff-induced evolving atherosclerotic lesion size is reduced by 55% after 21 days treatment (Chyu et al., 2004). Human studies indicate that EGCG can maintain cardiovascular health, but the evidence is inconclusive regarding the effectiveness for cardiovascular disease prevention or treatment (Arab et al., 2009; Wolfram, 2007). The major problems are its low stability, bioavailability and targeting specificity in humans or research animals (Chen et al., 1997; Lee et al., 2002; Warden et al., 2001). The blood peak concentrations of green tea catechins appear at 2 to 4 hours after oral administration. The absolute oral bioavailability of EGCG after drinking tea containing catechins at 10 mg/kg body weight is about 0.1% in humans and research animals (Lambert and Yang, 2003; Warden et al., 2001). The peak plasma EGCG concentration is 0.15 μM after drinking 2 cups of green tea (Lee et al., 2002). Moreover, EGCG is unstable in both water and physiological fluid in vitro (Barras et al., 2009; Lambert et al., 2003). EGCG stability is decreased by various metabolic transformations including methylation, glucuronidation, sulfation and oxidative degradation in vivo (Dou, 2009; Lu et al., 2003a; Lu et al., 2003b; Vaidyanathan and Walle, 2002). EGCG cannot target to specific cells or tissues. Hence, there is a critical need to use biocompatible and biodegradable nanocarriers to increase EGCG stability, cellular bioavailability and target specificity.

Detailed experimental methods are as follows.

Cell Culture:

Human monocytic THP-1 cell line was purchased from the American Type Tissue Culture Collection (ATCC, Rockville, Md.) and grew in a complete medium according to ATCC instructions. The complete medium consisted of RPMI medium supplemented with 10% fetal bovine serum and 0.05 mM of 2-mercaptoethanol. The cells were incubated in a 5% CO₂ atmosphere at 37° C. THP-1 cells were differentiated into macrophages by incubating them with the complete medium containing 50 ng/ml PMA for 72 h.

Preparation of EGCG Loaded NLC and CSNLC:

EGCG encapsulated in nanostructured lipid carriers (NLCE) and void nanostructured lipid carriers (VNLC) were prepared by a novel phase inversion-based process. Briefly, soy lecithin (70 mg) was dissolved in chloroform and dried under a nitrogen evaporator and freeze-dried for more than 24 hours using a vacuum freeze-dry system. Then glycerin tripalmitate (50 mg), tricaprate (300 mg), Solutol HS15 (330 mg) and EGCG (20 mg) were added into freeze-dried lecithin, they formed a lipid mixture. An aqueous mixture was composed of NaCl and deionized water. First, oil and aqueous phase was heated to 85° C. and mixed together. Then, the mixture was treated with three temperature cycles from 70 to 85° C. under magnetic stirring. In the last cycle, when the mixture was cooled to 79° C. (1 to 3° C. lower than the beginning of the phase inversion zone), cold deionized water (0° C.) was added to the mixture. The fast cooling-dilution process resulted in NLCE formation. Afterward, a slow magnetic stirring was applied to the suspension for forty-five minutes. VNLC were synthesized by using the same method without adding EGCG. All steps in the preparation of VNLC, NLCE were performed under nitrogen to prevent EGCG degradation. Both NLCE and VNLC were concentrated by ultrafiltration using Millipore Amicon Ultra-15 centrifugal filters. The concentrated nanoencapsulated EGCG was separated from nonencapsulated EGCG using a Sephadex™ G-25 column (GE Healthcare Bio-Sciences Corp, Piscataway, N.J.). Then both NLCE and VNLC were coated with 6 mg/ml chitosan (Sigma, St. Louis, Mo.) using a magnetic stirrer for 40 min at 4° C. to form CSNLCE and void CSNLC (VCSNLC), respectively.

Confirmation of NLC Morphology, Particle Size and Zeta Potential:

NLCE and CSNLCE were resuspended in 1× phosphate buffered saline (1×PBS), and stained by 2% of uranyl acetate. Their size and morphology were measured by using 200 Kv Hitachi H-8100 analytical transmission electron microscope (TEM). The size, size distribution, and zeta potential were measured by using Brookhaven BI-MAS and ZetaPALS analyzer, respectively.

HPLC Analysis of EGCG:

EGCG was detected using a high-performance liquid chromatography (HPLC) system (Waters Corporation, Milford, Mass.) with a UV detector and a ZORBAX SB-C18.5 μm, 150*4.6 mm (Agilent,). The mobile phase was composed of 86% water, 12% acetonitrile, 2% ethyl acetate and 0.043% sulfuric acid, and the flow rate was 1 ml/minute. EGCG was detected at 254 nm.

EGCG encapsulation efficiency and EGCG loading content are determined based on the following:

Encapsulation efficiency (%)=(Weight of EGCG added−Weight of free EGCG)/Weight of EGCG added×100%

Loading content (%)=Weight of EGCG/Weight of nanoparticles

Stability Study of EGCG Loaded NLC, CSNLC and Native EGCG Under Different Conditions:

To determine the stability of NLCE, CSNLCE and native EGCG in a pH range 1-7.4, Hydrochloric acid was added into 200 mM phosphate buffered saline to obtain four different pH solutions (pH=1, 3, 5, 7.4). All samples were then dissolved in these solutions to obtain a final EGCG concentration of 100 μM. The solutions were stored in tightly closed vials and incubated in a 37° C. water bath. EGCG concentrations were measured at 0 h, 0.5 h, 1 h, 1.5 h, 2 h, 2.5 hand 3 h.

To determine the stability in different temperatures, NLCE, CSNLCE and native EGCG containing 100 μM of EGCG were dissolved in 1×PBS solution (pH 7.4) and incubated at 4° C., 22° C. and 37° C. for 14 d, 19 h and 3 h, respectively. To simulate the cell growth, stability was evaluated in the cell culture medium RPMI1640 at 37° C. with or without cells, and with or without superoxide dismutases (SOD, 5 U/ml).

EGCG Content in Macrophages Treated by 100 μM of Native EGCG, NLCE and CSNLCE Under Different Conditions:

Macrophages derived from THP-1 cells were treated with 100 μM of NLCE, CSNLCE and native EGCG in the complete medium with or without SOD (5 U/ml). After 2 or 4 h incubation at 4° C. or 37° C., cells were washed three times with ice-cold 1×PBS. The attached cells were scraped by 200 μl of 2% ascorbic acid (pH 3) and each well was washed with 200 μl of methanol, which was then combined with 2% ascorbic acid as a mixture. The volume of mixture was measured and internal standard epicatechin was added into the mixture. After one cycle of freezing and thawing, mixtures were sonicated for 2 min in an ice-cold bath using a sonicator (Branson, Inc.). The mixture was centrifuged at 10,000×g for 20 min at 4° C. The clear supernatant solution was collected and injected into the HPLC system. Precipitates were washed with 0.5 ml of deionized water to get rid of acid and dried in the chemical hood overnight. The dried cells were digested by 0.5 N NaOH. Total cellular protein levels were determined by using a bicinchoninic acid (BCA) kit (Pierce, Cramlington). Total cellular uptake was expressed as μg of EGCG per mg of protein.

In Vitro Release Study:

EGCG release from nanocarriers was performed in 1×PBS at pH 5 using a dialysis method.

Cell Viability Assay:

For cell viability study, 3×10⁴ cells suspended in 150 ul medium were plated into each well of a 96-well plate and differentiated for 72 h. The cells were then treated with 1×PBS (treatment 1), native EGCG (treatment 2), VCSNLC (treatment 3), CSNLCE (treatment 4), VNLC (treatment 5), NLCE (treatment 6) for 18 h. Three EGCG concentrations (5 μM, 10 μM and 20 μM) were tested among all treatments. After 18 h, cells were incubated with 3-(4,5-dimethylthiazol-2-yl)-2,5-diphenyltetrazolium bromide (MTT) reagent (0.5 mg/mL in 1×PBS) for 4 h at 37° C. Then MTT reagent was aspirated and Dimethyl sulfoxide (DMSO) was added to solubilize formazan products. After 10 min incubation, the absorbance in each well was measured at 562 nm and 690 nm on the BioTek ELx800™ absorbance microplate reader (BioTek, Winooski, Vt.). The background absorbance (690 nm) was subtracted from the 562 nm measurements.

Minimally Modified Low-Density Lipoprotein Preparation and Cholesterol Measurement:

LDL was isolated from human plasma by a sequential ultracentrifugation method. Minimally modified LDL was prepared by an adaptation of a previously described method. Briefly, human LDL was exposed to 2 mM-CuSO4 for 5 h, and oxidation was confirmed by measuring thiobarbituric acid-reactive substances. Macrophage cells were incubated with the following treatments with or without 40 mg protein/ml of minimally modified-LDL: 1×PBS (treatment 1), native EGCG (treatment 2), VCSNLC (treatment 3), CSNLCE (treatment 4), VNLC (treatment 5), NLCE (treatment 6) containing 10 μM of EGCG for 18 h. After cellular lipid extraction, non-esterified/freecholesterol (FC) and total cholesterol (TC) were measured using a HPLC system. Stigmasterol was chosen as internal standard in both free cholesterol and total cholesterol measurement. Delipidated cellular protein levels were determined using a BCA kit. Esterified cholesterol (EC) was calculated as the difference between TC and FC and expressed as μg of cholesterol per g of protein.

Exemplary results are as follows.

Physical Characteristics of Nanoparticles:

Selected physical characteristics of nanoparticles are summarized in Table 3 and Table 4.

TABLE 3
Characteristics of nanoparticles¹.

| Nano- | Effective | Poly- | Zeta |
|---|---|---|---|
| particles | diameter (nm) | dispersity | potential (mV) |
| NLCE | 49.1 ± 1.1 | 0.26 ± 0.04 | −3.2 ± 4.54 |
| VNLC | 43.1 ± 3.3 | 0.31 ± 0.03 | −8.9 ± 2.96 |
| CSNLCE | 53.1 ± 1.5 | 0.24 ± 0.02 | 15.3 ± 1.78 |
| VCSNLC | 48.0 ± 0.7 | 0.25 ± 0.03 | 20.9 ± 0.43 |

¹Values are means ± SD, n = 3.

TABLE 4
Particle size, Zeta potential, and polydispersity of nanocarriers dissolved
in 1 X PBS at pH 7.4 at 4° C. and 37° C.².

| Nanoparticles | | Particle size (nm) | | Zeta potential (mV) | | Polydispersity | |
|---|---|---|---|---|---|---|---|
| Temperature | | 0 day | 50 days | 0 day | 50 days | 0 day | 50 days |
| 4° C. | NLCE | 46.3 ± 1.4 | 51.8 ± 1.8 | −3.57 ± 3.17 | −12.8 ± 0.23 | 0.185 ± 0.01 | 0.181 ± 0.02 |
| | CSNLCE | 53.5 ± 1.6 | 70.6 ± 0.5 | 13.25 ± 1.02 | 12.95 ± 4.32 | 0.194 ± 0.01 | 0.285 ± 0.01 |
| | | 0 hour | 10 hours | 0 hour | 10 hours | 0 hour | 10 hours |
| 37° C. | NLCE | 63.3 ± 1.3 | 94.7 ± 1.07 | −13.7 ± 7.8 | −7.3 ± 2.7 | 0.21 ± 0.01 | 0.344 ± 0.04 |
| | CSNLCE | 70.9 ± 2.2 | 108.9 ± 1.9 | 9.89 ± 4.0 | 13.2 ± 5.48 | 0.31 ± 0.01 | 0.373 ± 0.02 |

²Values are means ± SD, n = 3.

The sizes of all nanocarriers were less than 80 nm. CSNLCE had larger size than VNLC and NLCE. Both VCSNLC and CSNLCE were positively charged, but VNLC and NLCE had negative charge. EGCG encapsulation efficiency in NLC and CSNLC was about 90%. The EGCG loading content in NLC was around 3% respectively. Both NLCE and CSNLCE were spherical observed using a transmission electron microscope (TEM) (FIG. 6).

Stability of NLCE, CSNLCE and Native EGCG:

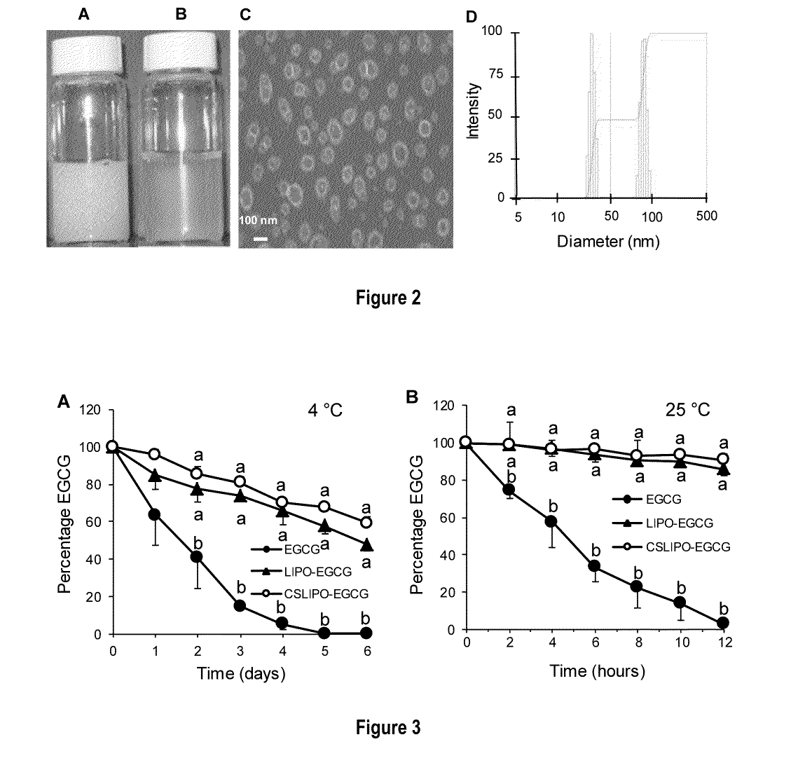

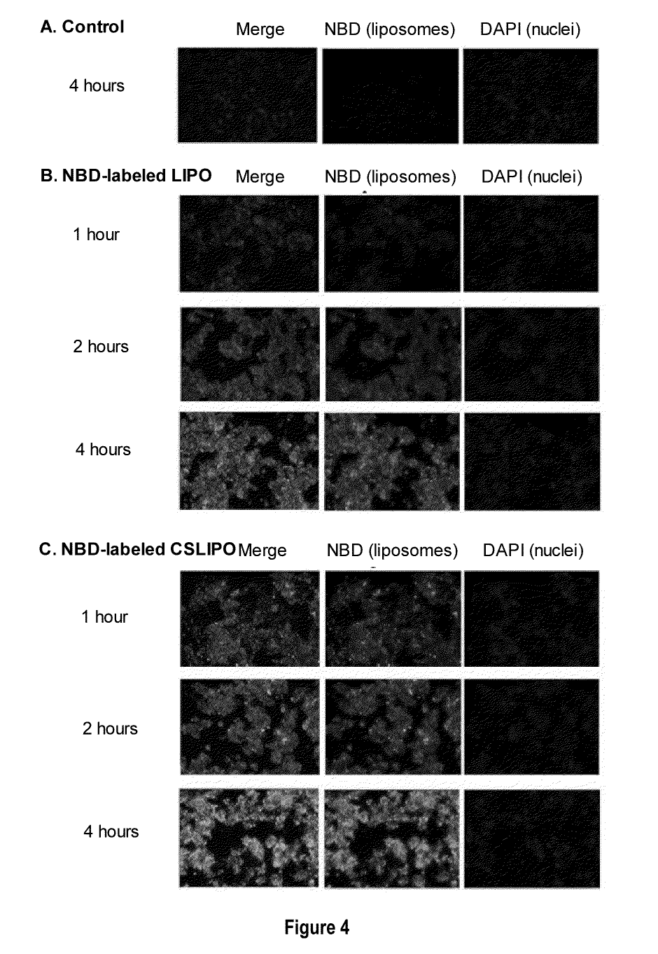

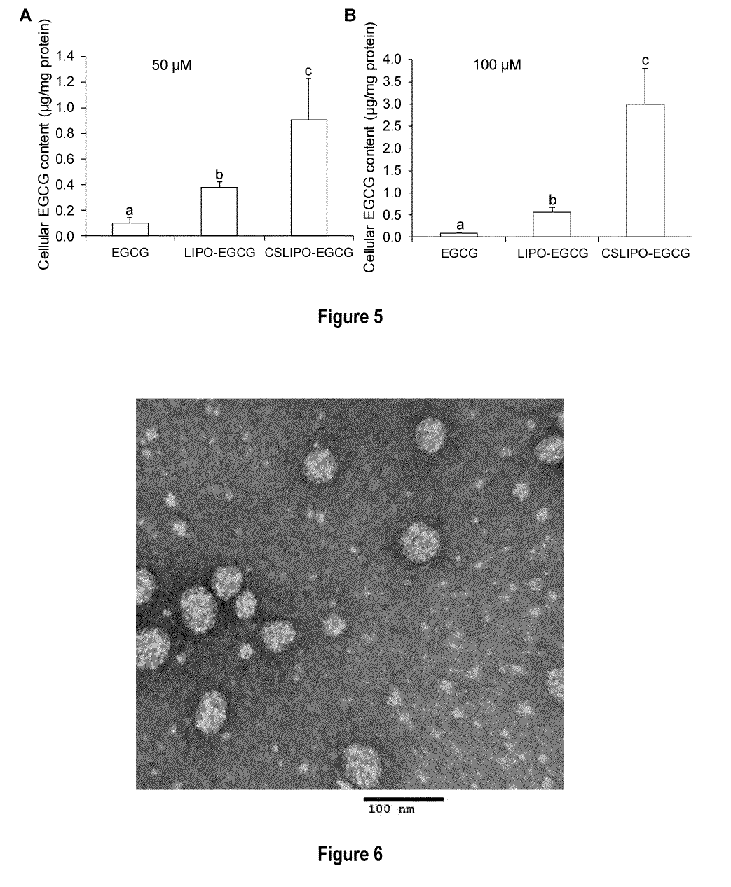

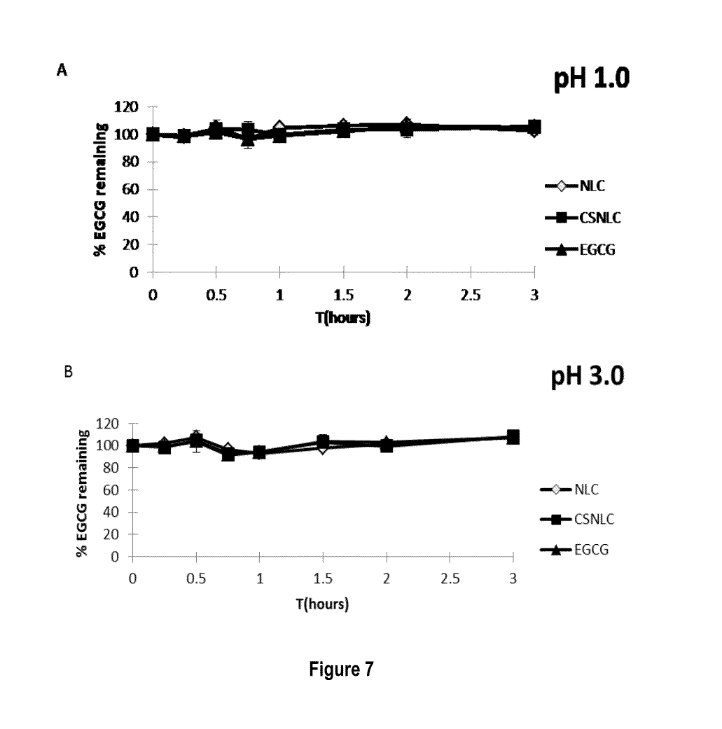

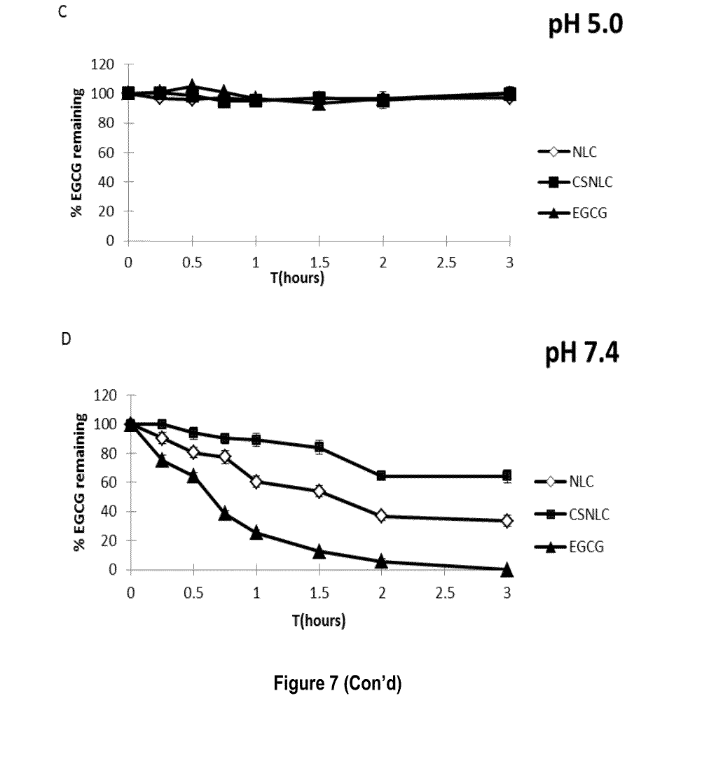

The stability of 100 μM of native EGCG, nanoencapsulated EGCG (NLCE and CSNLCE) were measured at different pH and temperatures. The data summarized in the FIG. 2-5 showed that the degradation of native EGCG and EGCG in nanoparticles were primarily dependent on the solution pH and temperature. In all conditions, the stability of EGCG at the same pH was in the following order: CSNLCE>NLCE>native EGCG. NLCE, CSNLCE and native EGCG at 100 μM concentration were stable in the acidic pH ranging from 1.0 to 5.0 at 37° C. for 3 h. There was no significant difference among them at this pH range. In the neutral pH 7.4, native EGCG (100 μM) was not stable and could not be detected at 37° C. after 3 h. The degradation rate of NLCE and CSNLCE were much slower compared to native EGCG at pH 7.4 at 37° C. After 2 h incubation, NLCE and CSNLCE containing 100 μM of EGCG were 7 and 12 times more stable than native EGCG, respectively (FIG. 7).

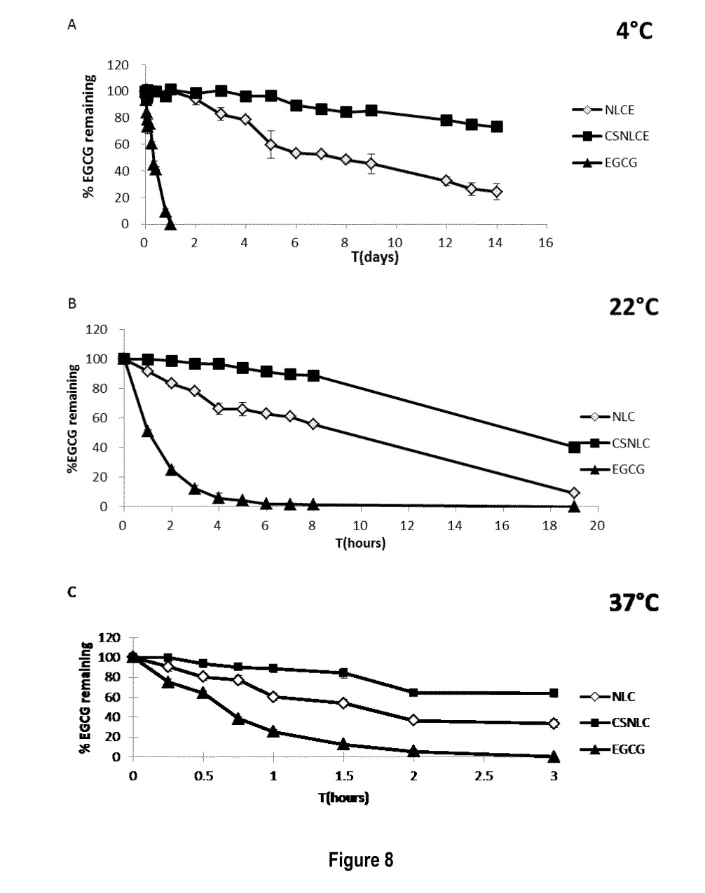

As the temperature decreased, the EGCG stability was increased. The stability of nanoparticles increased significantly compared with pure EGCG among 4, 22, 37° C. At 37° C., 100 μM of native EGCG were completely degraded after 3 h, however, NLCE 33.28% left (compared with EGCG P<0.01), and CSNLCE 64.18% left (compared with NLCE P<0.01). At 4° C., 91% of native EGCG was degraded after 19 hours whereas the degradation rate of EGCG in NLCE and CSNLCE was less than 4% at the same initial concentration. After incubating for 8 h at 23° C., CSNLCE concentration (89% left) was 58 times higher than native EGCG (1.52% left) (FIG. 8). In addition, high concentration of nanoparticles can be kept at 4° C. for a long period of time without obvious degradation. If 3000 μM NLCE and CSNLCE were kept at 4° C. for 50 days, 92% NLCE and 82% CSNLCE were detected and remained.

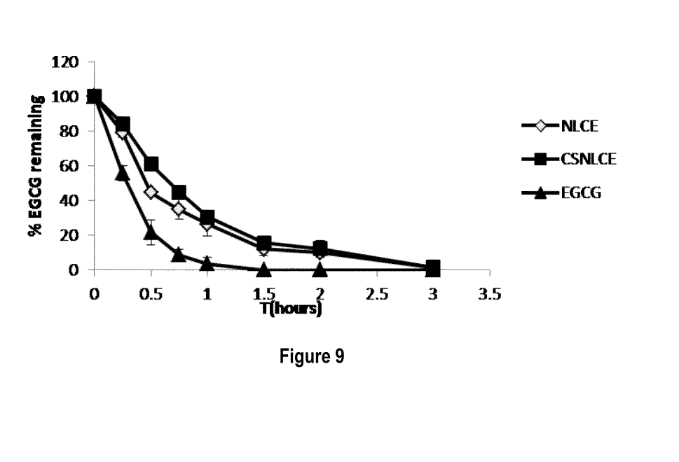

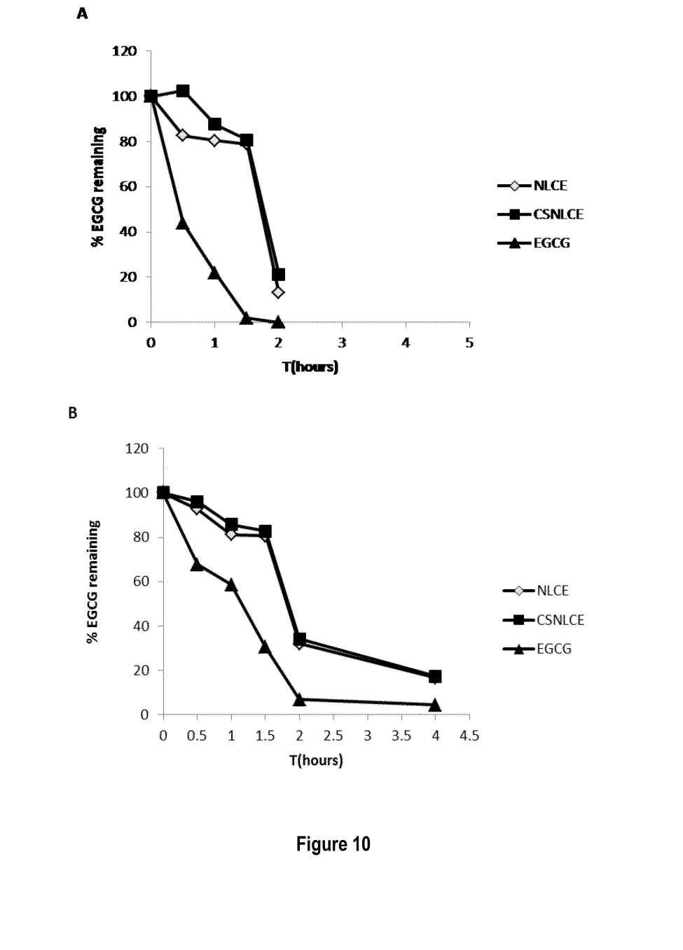

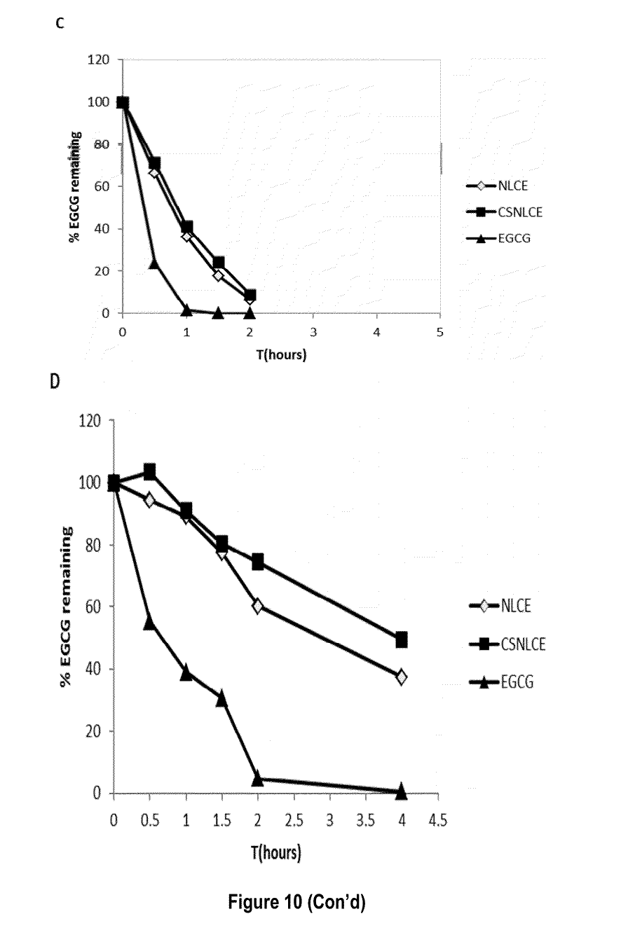

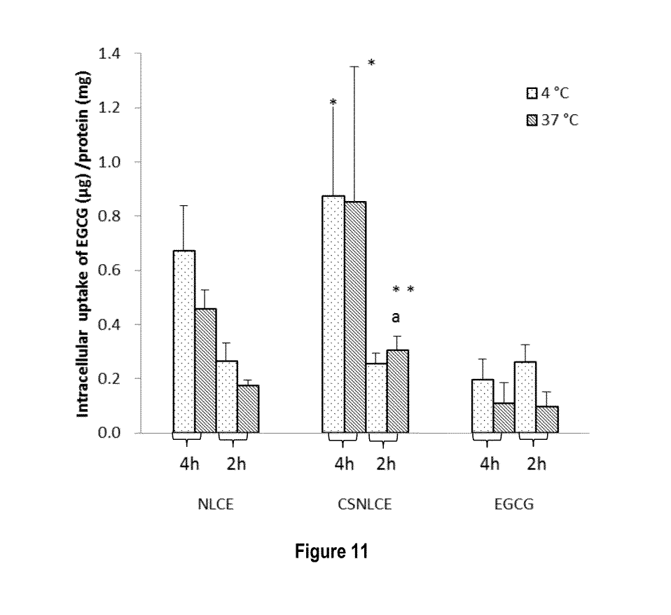

Nanoparticles could also prolong EGCG stability in cell culture medium RPMI1640 at 37° C. in the presence or absence of THP-1 derived macrophages. In RPMI1640 at 37° C., the stability of nanoparticles and EGCG decreased faster than in the 1×PBS and nanoparticles were much stable than EGCG. After 1 h incubation, 31% CSNLCE and 27% NLCE left compared with only 3.7% EGCG left. SOD (5 U/ml) can increase the stability of EGCG significantly than nanoparticles in the RPMI1640 at 37° C. With the macrophage cells exist, cells can increase stability of nanoparticles and EGCG slightly. In addition, SOD (5 U/ml) can increase stability of nanoparticles and native EGCG significantly in RPMI at 37° C. with macrophage cells exist (FIG. 9, 10).

EGCG Content Taken by THP-1 Macrophages:

After 2 h incubation at 37° C., the EGCG content in THP-1 derived macrophages treated by 100 μM of native EGCG, NLCE and CSNLCE in the complete medium without SOD was 0.031, 0.096, 0.14 μg/mg protein, respectively. Both nanocarriers increased cellular EGCG content significantly than native EGCG at the same treatment concentration.

Adding SOD into the culture medium significantly increased the cellular EGCG content in all treatments, and this improvement was time-dependent. After 2 h incubation at 37° C., the EGCG content in macrophages treated by 100 μM of native EGCG, NLCE and CSNLCE in the complete medium containing 5 U/ml of SOD was 0.098, 0.176, 0.307 μg/mg protein, respectively. After 4 h, the EGCG content in native EGCG, NLCE and CSNLCE treated macrophages in the presence of SOD was 0.109, 0.458, 0.853 μg/mg protein, respectively (FIG. 11). Cellular EGCG content in CSNLCE treated macrophages was significantly higher than native EGCG (p<0.01) and (p<0.05) with 2 h incubation at 37° C. In the condition of 4 h incubation in presence of SOD at 4° C. and 37° C., Cellular EGCG content in CSNLCE treated macrophages was significantly higher than native EGCG (p<0.05).

Cell Viability Assay:

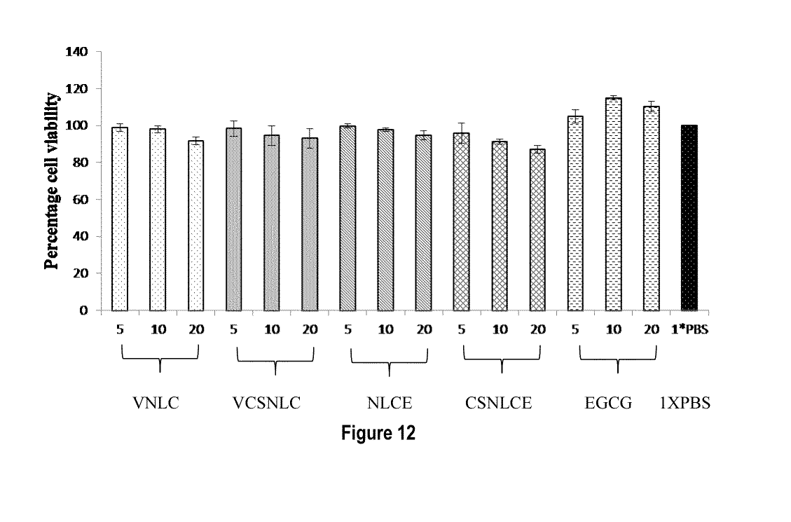

After treating THP-1 macrophage cells with 5, 10, and 20 μM of native or nanoencapsulated EGCG (NLCE and CSNLCE) and responsive void nanocarriers (VNLC and VCSNLC) for 18 hours, the cell viability was more than 90% in all treatments. (FIG. 12).

Effect of NLCE and CSNLCE on Cholesterol Accumulation:

Without adding oxLDL in the culture medium, nanoencapsulated EGCG can significantly decrease cellular esterified cholesterol (EC) content in the macrophages compared with control—1×PBS, native EGCG and void nanoparticles.

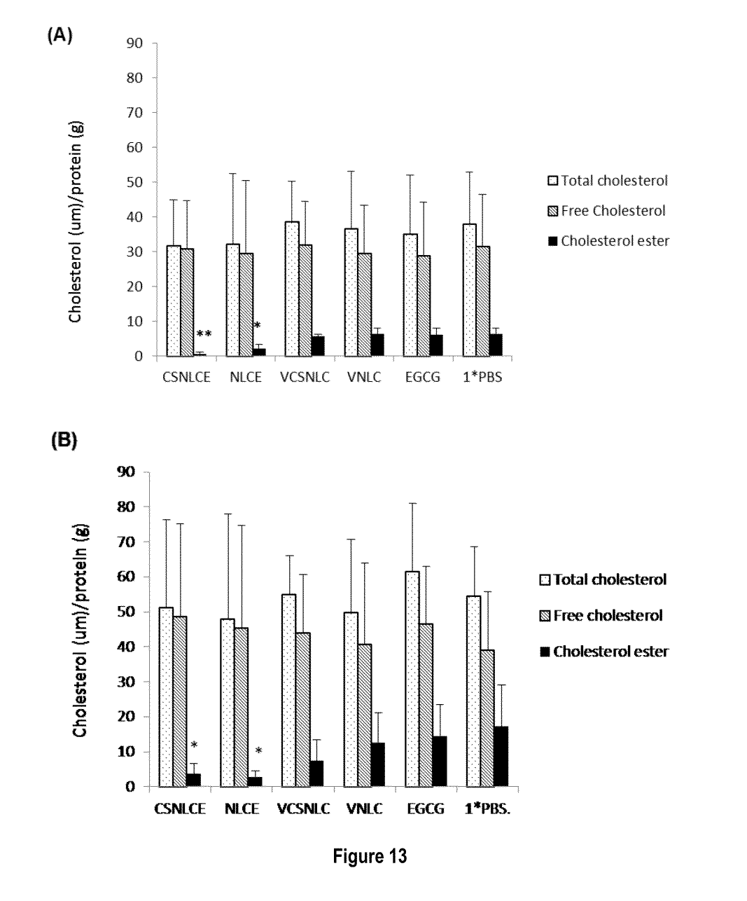

As compared to 1×PBS treatment, CSNLCE and NLCE decreased cellular EC content 10 times and 2.8 times respectively. In contrast with EGCG, CSNLCE and NLCE decreased cellular EG content 9.4 times and 2.7 times (FIG. 13 A).

After adding 40 mg protein/ml of minimally modified-LDL in the culture medium, NLCE and CSNLCE compared to native EGCG, decreased macrophage EC content by 5 and 4 times, respectively (FIG. 13 B).

Nanoparticle Uptake and Distribution by Fluorescent Microscopy:

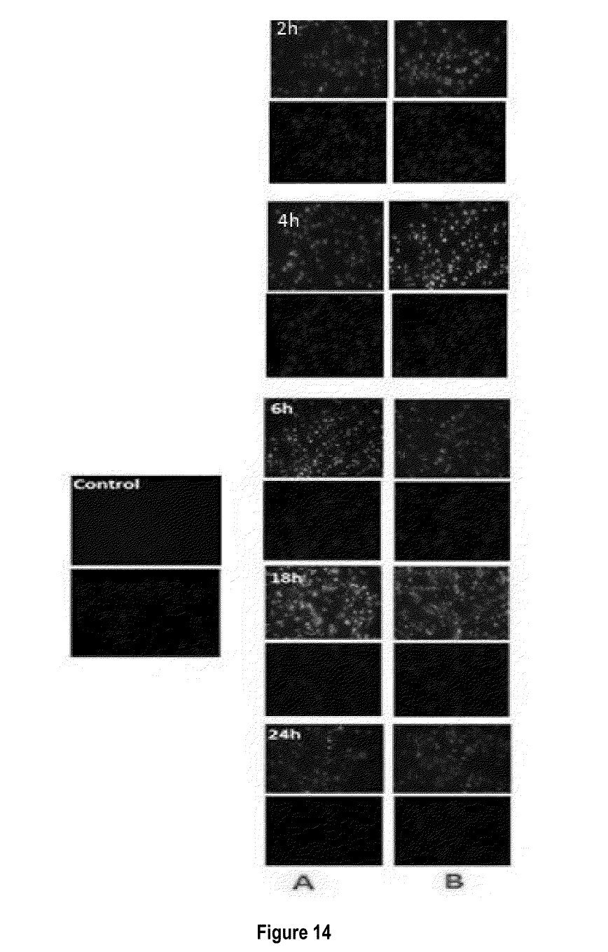

In the case of cells exposed to NLC and CSNLC formulations, highly diffused fluorescence was clearly observed in the entire intracellular matrix. From fluorescent microscopy images (FIG. 14), NLC, CSNLC lipid carriers have been taken up significantly, and in 18 h, the signal is highest so that the amount should be the most.

### Example 4

### KDdiA-PC Increased the Binding Affinity of Nanoliposomes to Macrophages

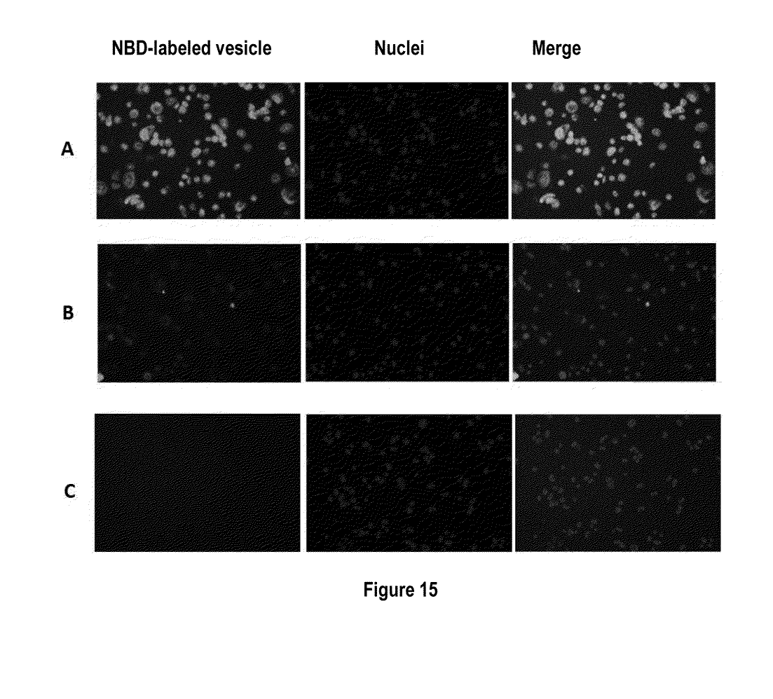

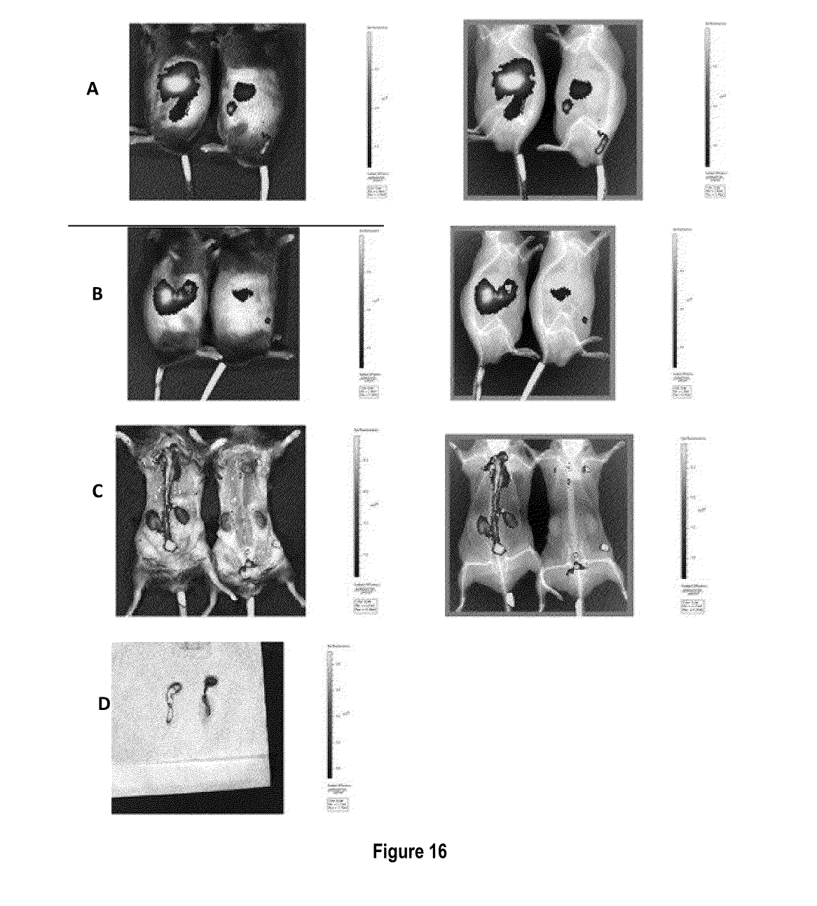

FIGS. 15A-C illustrates results of binding assays of liposomes to THP-1 derived macrophages. (Human monocytic THP-1 cell line was grew in a complete medium consisted of RPMI medium supplemented with 10% fetal bovine serum and 0.05 mM of 2-mercaptoethanol. The cells were incubated at 37° C., 95% humidity, and an atmosphere of 5%. 3×10⁴ THP-1 cells were seeded into each well of 96-well sterile flat-bottomed plates, and were differentiated into macrophages by incubating them with the complete medium containing 50 ng/ml PMA for 72 h).

Macrophages derived from THP-1 cells were treated with fluorescence-labeled liposome vesicle (5 μg/ml of lipid concentration, 120 nm of mean vesicle particle size) in PRMI 1640 containing 2% lipid free serum incubated for 4 hour at 37° C. (See FIG. 15). FIG. 15A indicates that Ligand-liposome (composed of 30 mol % KDdiA-PC) showed very strong binding to macrophages. FIG. 15B suggests that the control-liposome without KDdiA-PC showed much weaker binding to macrophage, only slightly better than the 1×PBS control (FIG. 15C). Liposomes were labeled with 7-Nitro-2-1,3-benzoxadiazol-4-yl (NBD)-PE (1.0 mol % relative to the total lipid) as fluorescence dye (λ of excitation is 460 nm, λ of emission is =535 nm) (green color). Cell nuclei were stained by DAPI (λ of excitation is 358 nm, λ of emission is 461 nm) (blue color).

Liposomes with KDdiA-PC have significantly higher binding affinity to macrophages and results in more uptake by macrophages compared to liposomes without KDdiA-PC.

### Example 5

### KDdiA-PC Increased the Target Specificity of Liposomes to Atherosclerotic Lesion in LDL Receptor Null Mice

FIGS. 16A-D show that KDdiA-PC-liposomes target to atherosclerosis in LDL receptor null mice (a well-known atherosclerosis animal model). Male low-density lipoprotein receptor-deficient (LDLr−/−) mice (C57BL6 background) were fed with Harlan Teklad an atherogenic diet (TD.88137) containing 21% of saturated fat (w/w) and 0.15% of cholesterol (w/w) for 24 weeks from 6 weeks old. Mice developed atherosclerotic lesions on aortic arch and abdominal arterial after feeding this atherogenic diet for 24 weeks. Mice were housed at 22 to 24° C., 45% relative humidity and a daily 10/14 light/dark cycle with the light period from 06:00 to 16:00. Food and water were given ad labium. Body weights of mice at the time of experiments were 40-45 g.

To arteriosclerosis model mice, the KDdiA-PC containing liposome vesicles and control liposome vesicles, which were labeled with 1,1′-dioctadecyl-3,3,3′,3′-tetramethyl indotricarbocyanine iodide (DiR) near infrared (NIR)fluorescence dye (λ of excitation is 730 nm, λ of emission is 790 nm), were intravenously injected through tail vein. Twenty hours later, NIR images combined with X-ray images were obtained from the left side (A) and right side (B), or after exposing the aorta by cutting the abdomen open (C) and isolated aorta from each mouse (D) using an IVIS® Lumina XR imaging system (See FIG. 16). KDdiA-PC significantly and dramatically increased the binding affinity and target specificity of liposomes to atherosclerotic lesions.

### Example 6

### KOdiA-PC Increased the Target Specificity of Liposomes to Atherosclerotic Lesion in LDL Receptor Null MiceLiposomes Preparation

After soy phosphotidylcholine (>95%) was dried under nitrogen and resuspended into 1×PBS (pH 7.4), the suspension was passed through 0.2 μm polycarbonate filter followed by 0.08 μm polycarbonate filter (10 times for each filter) using an Avanti Mini-Extruder Set (Avanti Polar Lipids, Inc., Alabaster, Ala.) to synthesize small unilamellar liposomes. The targeted liposomes were prepared by replacing 30 mol % of soy lecithin with KOdiA-PC. For in vitro binding experiments, 2 mol % of the fluorescent dye, 7-nitro-2-1,3-benzoxadiazol-4-yl-phosphotidylcholine (NBD-PC), was added to soy phosphotidylcholine. For in vivo imaging experiments, 2 mol % of the near-infrared fluorescent dye, 1,1′-dioctadecyl-3,3,3′,3′-tetramethyl indotricarbocyanine iodide (DiR), was added to soy phosphotidylcholine.

### Vitamine E Nanocarrier (NLC) Preparation

Soy phosphotidylcholine (>95%), ethoxylated stearic and oleic acid ester, Vitamin acetate, EGCG and acetic acid were dissolved into ethanol and dried down under nitrogen. Hot deionized water (80° C.) was added into the dried mixture. The suspension was homogenized for 1 minute followed by sonication for 8 minutes. The size and distribution of EGCG encapsulated lipid nanocarriers (NLC-EGCG) were measured by means of Brookhaven BI-90 particle size analyzer. For in vitro binding experiments, 2 mol % of the fluorescent dye, 7-nitro-2-1,3-benzoxadiazol-4-yl-phosphotidylcholine (NBD-PC), was added to the mixture. For in vivo imaging experiments, 2 mol % of the near-infrared fluorescent dye, 1,1′-dioctadecyl-3,3,3′,3′-tetramethyl indotricarbocyanine iodide (DiR), was added to the mixture. Macrophages derived from THP-1 cells were treated with NBD-labeled NLC-EGCG with or without KOdiA-PC in PRMI 1640 for 2 hour at 37° C. The binding affinity was determined using a fluorescence, and cellular EGCG content was measured using a HPLC system. Total cellular protein levels were determined by using a bicinchoninic acid (BCA) kit (Pierce, Rockford, Ill.). Cellular EGCG content was expressed as μg of EGCG per mg of protein.

### Elicited Peritoneal Macrophage Isolation

C57BL6J mice were given an intraperitoneal injection of 1.0 mL Brewer thioglycollate broth (4.05 g/100 mL). After three days, elicited peritoneal cells were collected by peritoneal lavage with 10 mL of cold Ca2+- and Mg2+-free Hanks' balanced salt solution four times from the peritoneal cavity. Peritoneal fluid is collected into sterile tubes and immediately centrifuged at 600 g at 4° C. for 15 minutes. Cell pellet was resuspended in RPMI 1640 medium supplemented with 10 mM HEPES, 100 units/mL penicillin, and 100 μg/mL streptomycin, and 10% fetal bovine serum. The cells were plated on 24-well tissue culture plates (1.25×105 per well) and allowed to adhere at 37° C. in a CO2 incubator for 2 hours. Nonadherent cells were removed by washing with sterile 1×PBS. Adhesive cells were maintained in RPMI 1640 medium containing 10% FBS, 100 units/mL penicillin, and 100 μg/mL streptomycin at 37° C.

### In Vitro Binding Assay

Human monocytic THP-1 cell line was purchased from the American Type Tissue Culture Collection (ATCC, Manassas, Va.) and cultured in the RPMI1640 medium following to ATCC instructions. THP-1 cells were differentiated into macrophages by incubating with 50 ng/mL PMA for 72 hours. The THP-1 derived macrophages were treated with 1×PBS, NBD-labeled liposomes (or nanocarriers) without target ligands (oxidized phospholipids), NBD-labeled liposomes (or nanocarriers) with target ligands (oxidized phospholipids) dissolved in 1×PBS (pH 7.4) for 2 hours at 4° C. Cellular binding of targeted and untargeted liposomes (or nanocarriers) were observed under a fluorescence microscopy. Microscopy settings were identical for all measures to allow equal comparison of the images. Fluorescence intensities were quantified using the NIH imageJ software.

### Competitive Binding Assay

For competitive study, mouse peritineal macrophages were incubated as follows: 1) NBD-labeled liposomes without target ligands (oxidized phospholipids); 2) NBD-labeled liposomes with target ligands (oxidized phospholipids); 3) NBD-labeled liposomes without target ligands and RPE-labeled anti-CD36 antibody; 4) NBD-labeled liposomes with target ligands and RPE-labeled anti-CD36 antibody. All cells were incubated for 2 hours at 4° C. After incubation, macrophages were rinsed three times with ice cold 1×PBS (pH 7.4) and fixed with 3.7% formaldehyde in 1×PBS (pH 7.4) for 10 minutes at room temperature. After washing with ice cold 1×PBS (pH 7.4) three times, nuclei were stained with DAPI solution (IHC world) for 10 minutes at room temperature in the dark. Cells were washed again with cold 1×PBS (pH 7.4) and visualized under a fluorescent microscope.

For knockdown assays, CD36 siRNA (Life Technologies) was used to knock down CD36 expression in mouse peritoneal macrophages isolated by the above method, following manufacturer's instructions. A CD36 negative control siRNA (Life Technologies) was also used. After 48-hour transfection, the macrophages were treated with NBD-labeled liposomes without target ligands (oxidized phospholipids) or NBD-labeled liposomes with target ligands (oxidized phospholipids) for 2 hours at 4° C. After incubation, cells were rinsed three times with ice cold 1×PBS (pH 7.4) and fixed with 3.7% formaldehyde in 1×PBS (pH 7.4) for 10 minutes at room temperature. After washing with ice cold 1×PBS (pH 7.4) three times, nuclei were stained with DAPI solution (IHC world) for 10 minutes at room temperature in the dark. Macrophages were washed again with ice cold 1×PBS (pH 7.4) and visualized under a fluorescent microscope. CD36 expression in CD36 knockdown macrophages were also measured using CD36 antibodies. Briefly, macrophages were washed with 1×PBS and fixed with cold methanol for 10 minutes. After incubation with 1% BSA for 1 hour at room temperature, the cells were stained with RPE-conjugated rat anti-mouse CD36 antibody (1:200) overnight at 4° C. in the dark. After CD36 staining, nuclei were stained with DAPI solution (IHC world) at room temperature in the dark. Macrophages were visualized under a fluorescent microscope.

All experiments were done in triplicate with different preparations of cells. Microscopy settings were identical for the different incubations to allow comparison of the results.

### In Vivo Targeting of Ligand to Atherosclerotic Plaque

The animal protocol was approved by the institute of animal care and use committee of Texas Tech University. Male low-density lipoprotein receptor-deficient (LDLr−/−) mice (C57BL6 background) were fed with Harlan Teklad an atherogenic diet (TD.88137) containing 21% of saturated fat (w/w) and 0.15% of cholesterol (w/w) for 20 to 30 weeks. Mice were housed at 22 to 24° C., 45% relative humidity and a daily 10/14 light/dark cycle with the light period from 06:00 to 16:00. Food and water were given ad libitum. Mice were paired based on their body weight. One mouse was treated liposomes with targeted ligands (oxidized phospholipids), and the other mouse was treated with liposomes without targeted ligands in each paired group.

For measuring the target specificity of liposomes carrying ligands (KOdiA-PC) to arteriosclerosis model mice, targeted and untargeted liposomes were labeled with 1,1′-dioctadecyl-3,3,3′,3′-tetramethyl indotricarbocyanine iodide (DiR), a near infrared (NIR) fluorescence dye (λ of excitation is 730 nm, λ of emission is 790 nm). The DiR-labeled liposomes were intravenously injected into mice via tail veins. After 20-hour injection, mice were sacrificed and then hearts were perfused with PBS from the left ventricular. The fluorescence reflectance images of aortas were acquired in situ and after isolation in the imaging chamber of the IVIS® Lumina XR imaging system (Caliper Life Science, USA). Subsequently, the dissected hearts and aortas were embedded in Tissue-Tek O.C.T. (Sakura), snap-frozen in liquid nitrogen, and sectioned using a cryostat (5 um thick). DiR signals in the cross-sections of aortas were observed using a fluorescence microscope with a Cy7 filter. The adjacent cross-sections of aortas were stained with oil red O for identifying atherosclerotic lesions. Sections were visualized under a microscope.

### Results

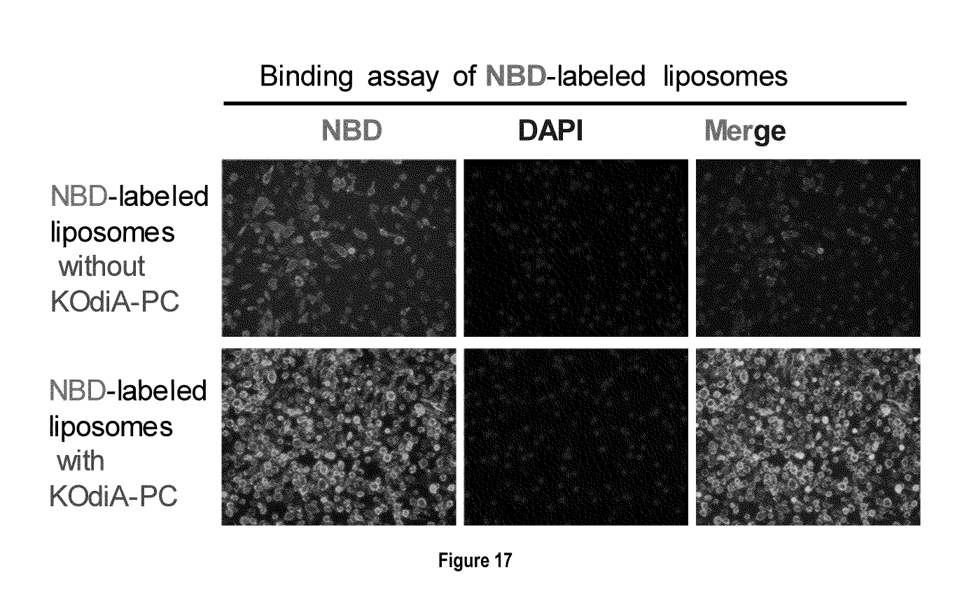

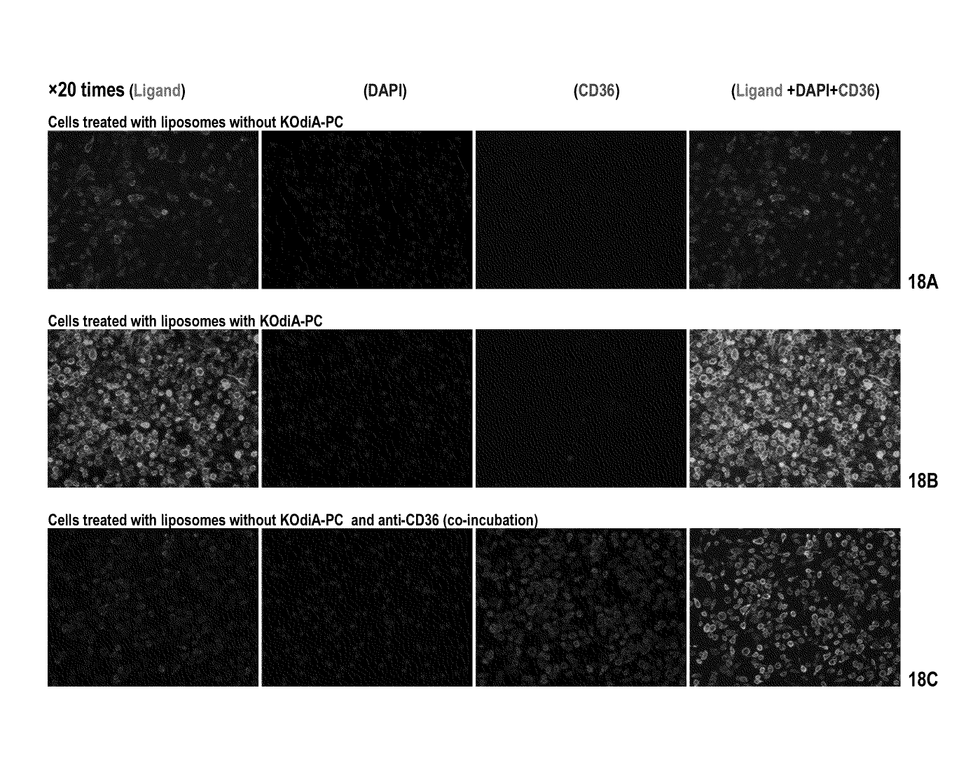

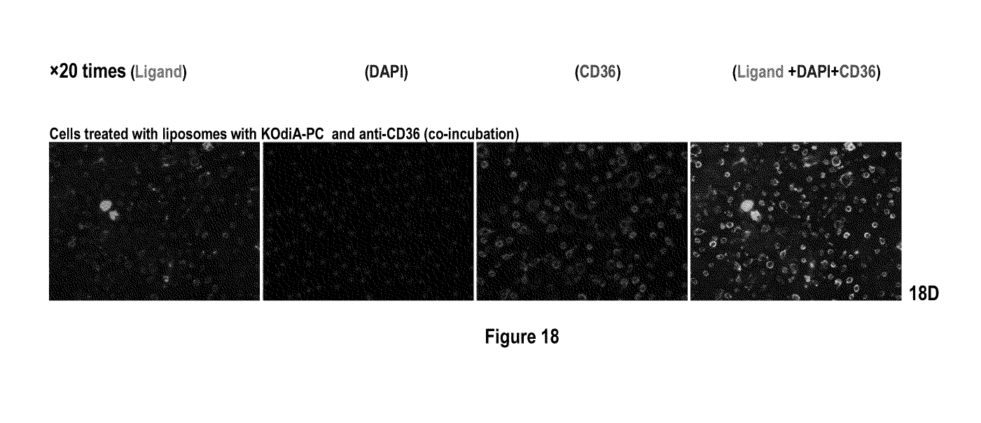

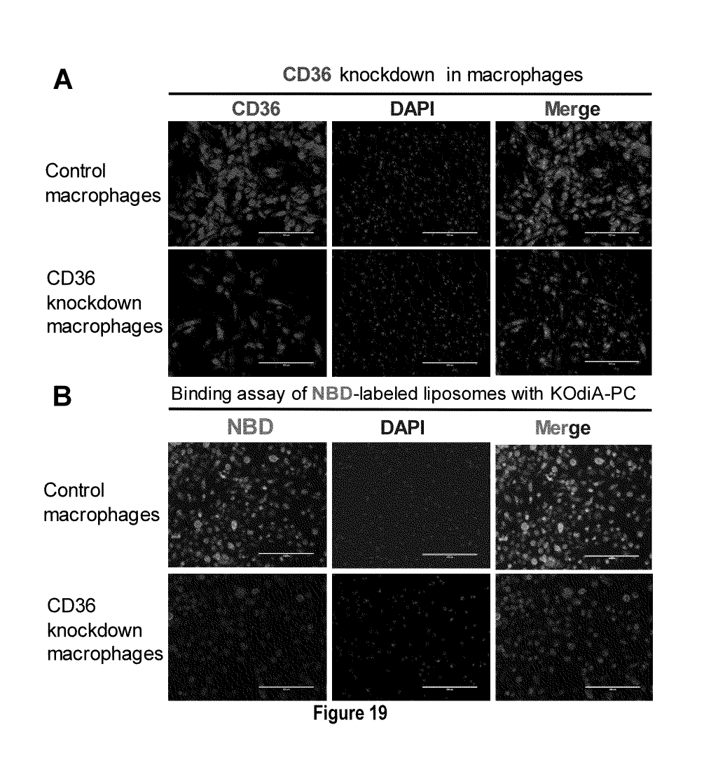

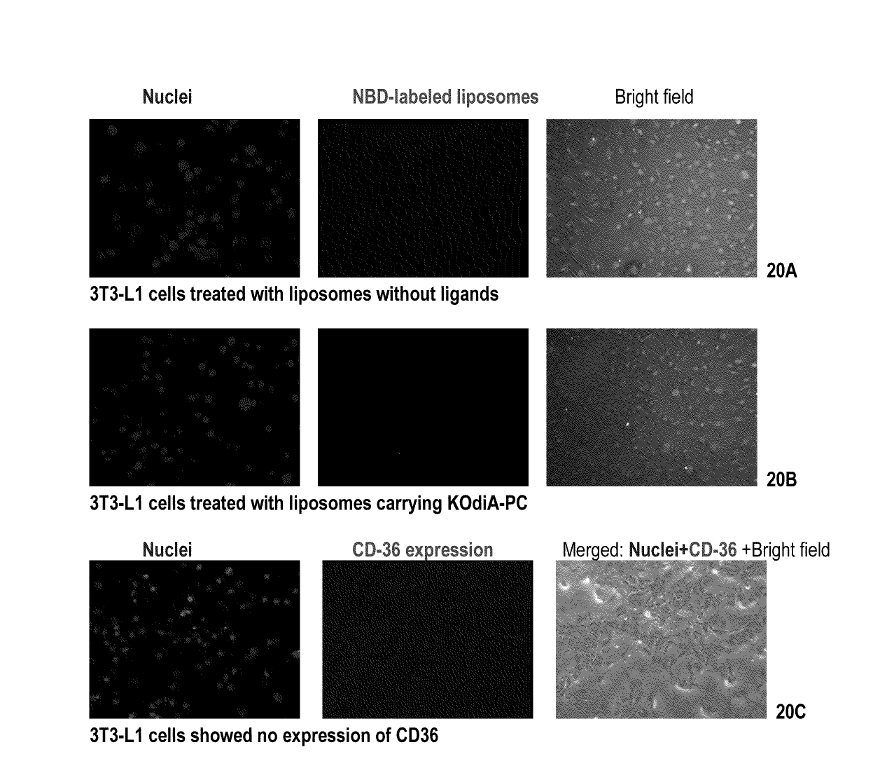

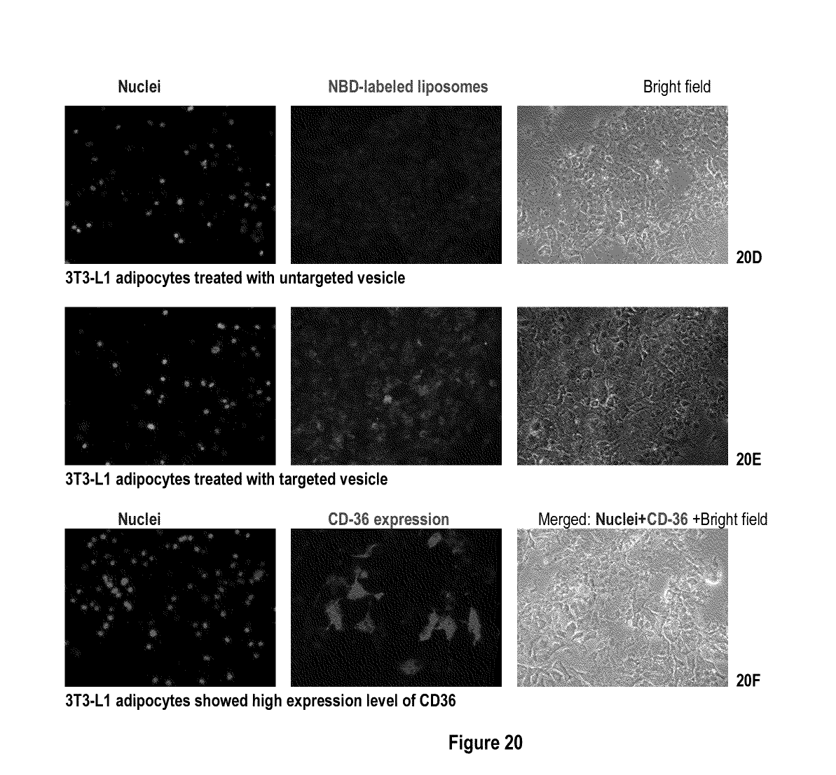

Macrophages derived from THP-1 cells were treated with NBD-labeled liposomes with or without target ligands (KOdiA-PC) for 2 hours at 4° C. (FIG. 17). NBD-labeled nanocarriers were green. Cell nuclei were stained by DAPI (blue color). Target ligands increased the binding affinity of liposomes to THP-1 derived macrophages. Liposomes carrying KOdiA-PC had higher binding affinity to mouse peritoneal macrophages than liposome not carrying KOdiA-PC (FIG. 18). Immunostaining of CD36 showed the expression of CD36 at high levels in mouse macrophage. The fluorescent targeted vesicle strongly bound to macrophages, whereas the untargeted vesicle did not. The binding of the fluorescent targeted vesicle to the cells was dramatically blocked by the adding of anti-CD36 antibody. Co-incubation with liposomes carrying KOdiA-PC and CD36 antibody inhibited liposomes binding to macrophages (FIG. 18). After knocking down CD36 in macrophages, the binding affinity of NBD-labeled liposomes with ligands was significantly decreased, indicating that targeted nanocarriers bound to macrophages via CD36 (FIG. 19). Negative control siRNA did not affect the expression of CD36 in macrophages and also the binding of targeted vesicle to the cells. Liposomes carrying KOdiA-PC had higher binding affinity to 3T3-L1 adipocytes (high expression of CD36) than 3T3-L1 preadipocytes (low expression of CD36) (FIG. 20).

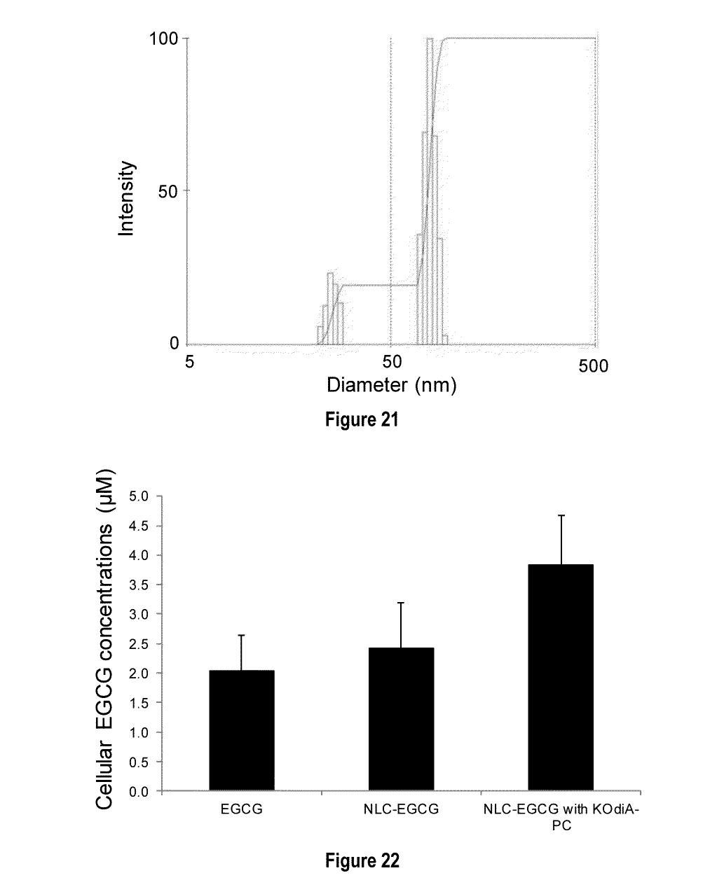

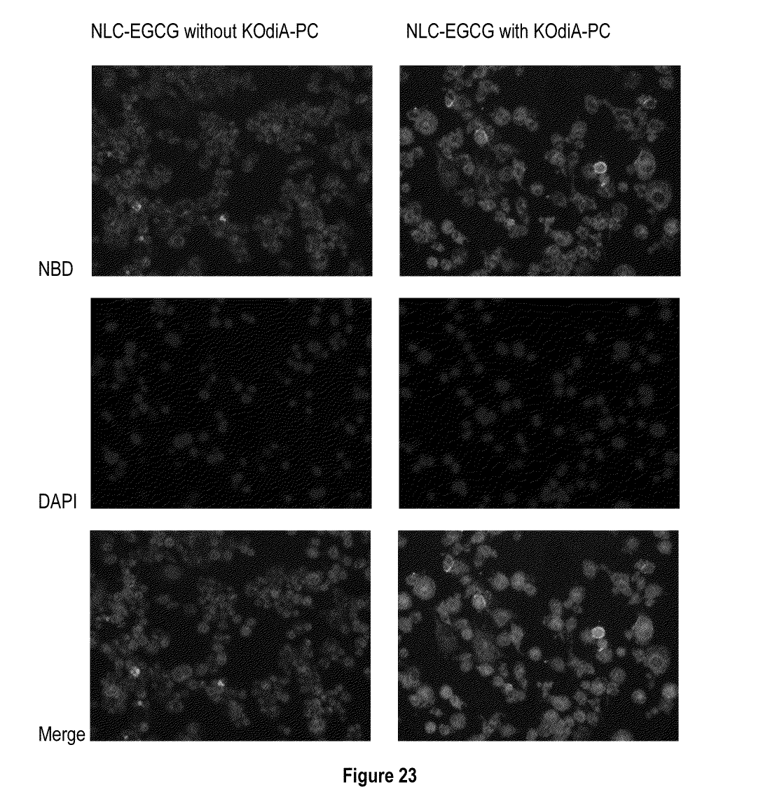

The size of NLC-EGCG was about 60 nm in diameter (FIG. 21). NLC-EGCG carrying KOdiA-PC increased macrophage EGCG content as compared to NLC-EGCG without ligand and free EGCG (FIG. 22). KOdiA-PC increased the binding affinity of NLC-EGCG to macrophages. NLC-EGCG carrying KOdiA-PC had significantly higher binding affinity to macrophages and resulted in more uptake of NLC-EGCG by macrophages compared to NLC-EGCG without KOdiA-PC, resulting higher macrophage EGCG content (FIG. 23).

### In Vivo Targeting Results:

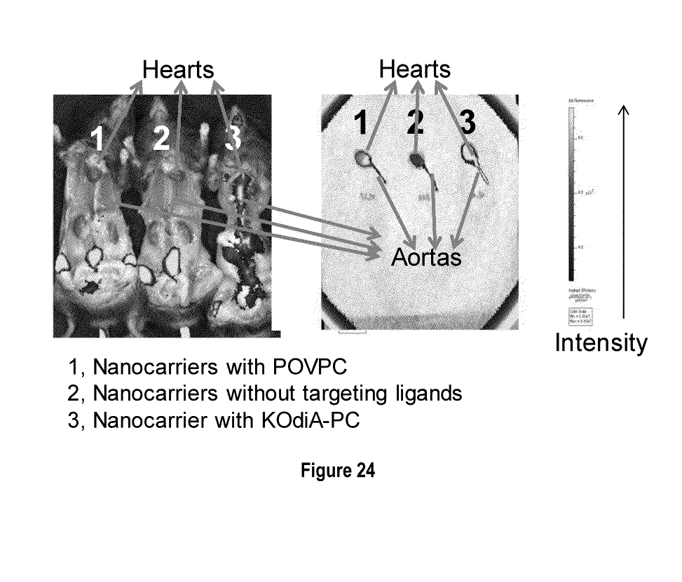

Liposomes carrying KOdiA-PC targeted to atherosclerotic aortas and lesions (FIG. 24). KOdiA-PC is better than POVPC in targeting to atherosclerotic lesions (FIG. 24). Nanocarriers containing KOdiA-PC, POVPC, or no ligands were injected into the mice via tail vein injection. All nanocarriers were labeled with 1,1′-dioctadecyl-3,3,3′,3′-tetramethyl indotricarbocyanine iodide (DiR), a near infrared (NIR) dye (λ of excitation is 730 nm, λ of emission is 790 nm). After 22 hours, mice were sacrificed and aortas were imaged using an IVIS® Lumina XR imaging system. The higher DiR intensity, the higher binding affinity of nanocarriers to aortas. The same nanocarriers were also injected into control mice (without atherosclerotic lesions), the nanocarriers containing KOdiA-PC did not target to aortas (data not shown). KOdiA-PC significantly and dramatically increased the binding affinity and target specificity of nanocarriers to atherosclerotic lesions.

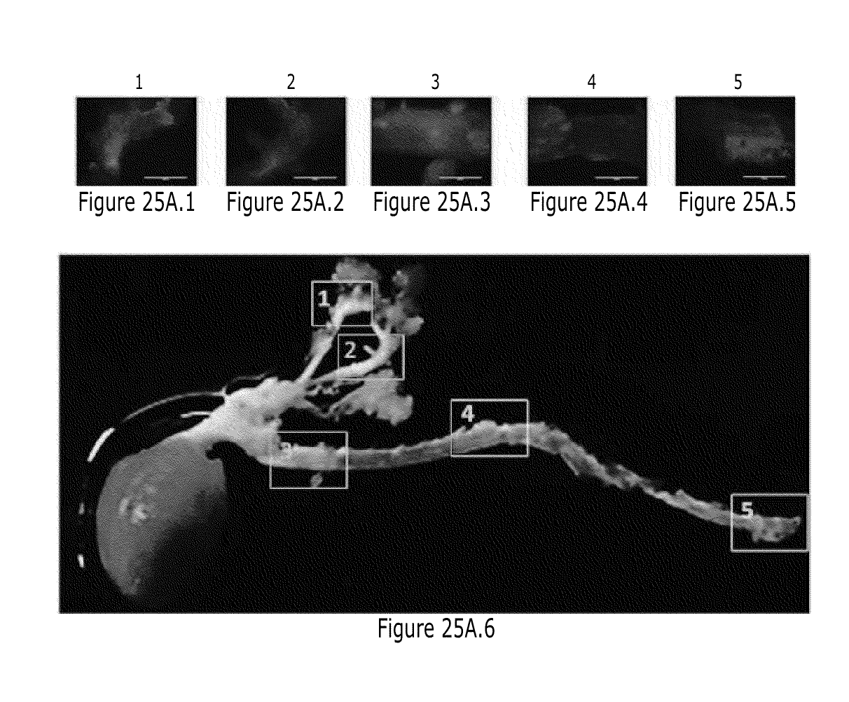

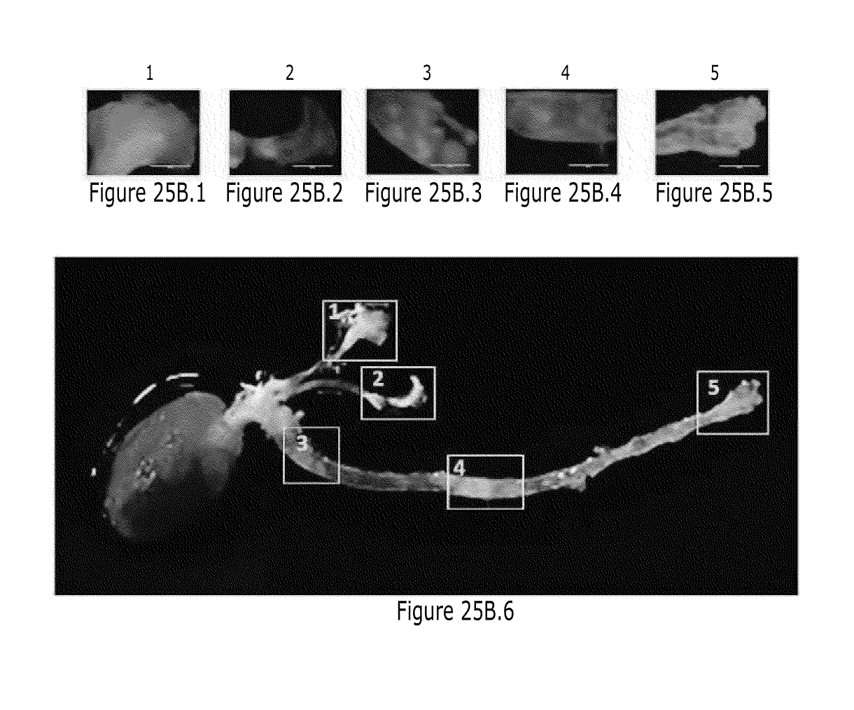

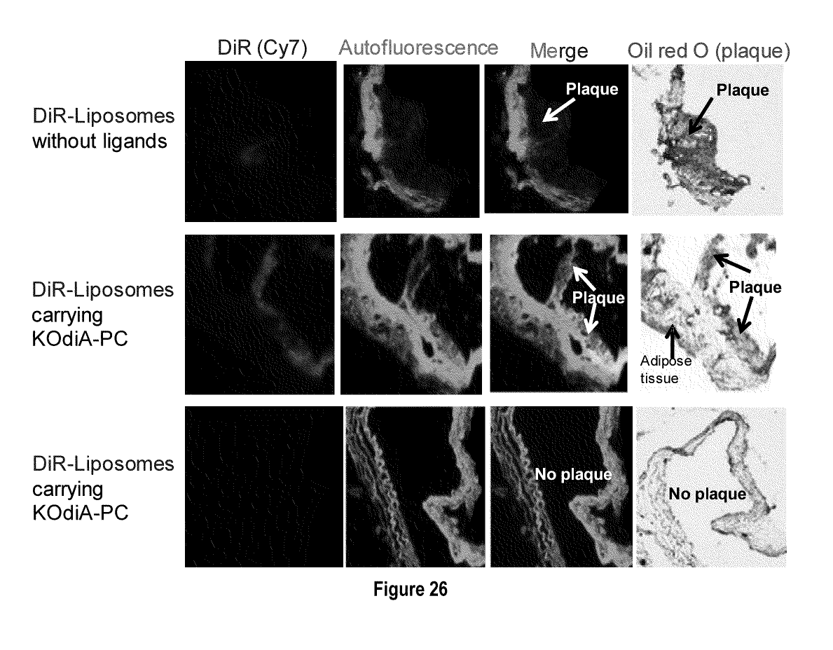

Liposomes carrying KOdiA-PC can target atherosclerotic lesion (FIG. 25) on the surface of aortas and the cross-sections of aortas. For analysis in FIG. 25, aortas were isolated from each treatment groups. The lesion was imaged using phase contrast and Cy7 filters. Black areas represent aortic lesions. Purple spectrum color represents nanocarrier accumulation. Representative images from the cross-sections of aortic arches are shown in FIG. 26.

### REFERENCES

Additional details concerning the background and other relevant information can be found in, for example, Arab, L., et al., 2009. Stroke. 40, 1786-1792; Barras, A., et al., 2009. Int J Pharm. 379, 270-277; Basu and Lucas, 2007. Nutr Rev. 65, 361-375; Berliner, J., et al., 1997. Thromb Haemost. 78, 195-199; Berliner, J. A., et al., 2009. J Lipid Res. 50 Suppl, S207-S212; Boullier, A., et al., 2001. Ann N Y Acad. Sci. 947, 214-223; Boullier, A., et al., 2000. J Biol Chem. 275, 9163-9169; Brown, M, et al., 1979. J Cell Biol. 82, 597-613; Chen, L., et al., 1997. Drug Metab Dispos. 25, 1045-1050; Chyu, K Y, et al., 2004. Circulation. 109, 2448-2453; Curtiss, L K, 2009. N Engl J Med. 360, 1144-1146; de Winther, M P, et al., 2000. Arterioscler Thromb Vasc Biol. 20: 290-297; Dou, Q P, 2009. Nutr Cancer. 61, 827-835; Febbraio, M., et al., 2000. J Clin Invest. 105, 1049-1056; Goldstein, J L, et al., 1979. PNAS USA. 76: 333-337; Harb, D., et al., 2009. Cardiovasc Res. 83:42-51; Hashimoto and Sakagami, 2008. Anticancer Res. 28: 1713-1718; Hayakawa, S., et al., 2001. Biochem Biophys Res Commun. 285: 1102-106; Heurtault, B., et al., 2002. Pharm Res. 19, 875-880; Ichikawa, D., et al., 2004. Biol Pharm Bull. 27, 1353-1358; Kunjathoor, V., et al., 2002. J Biol. Chem. 277: 49982-49988; Lambert, J D, et al., 2003. J Nutr. 133: 4172-4177 and 3262S-3267S; Lee, M J, et al., 2002. Cancer Epidemiol Biomarkers Prev. 11: 1025-1032; Leitinger, N., et al., 1999. PNAS USA. 96, 12010-12015; Lipinski, M J, et al., 2009. JACC Cardiovasc Imaging. 2: 637-647; Lu, H., et al., 2003a. Drug Metab Dispos. 31: 452-461; Lu, H., et al., 2003b. Drug Metab Dispos. 31, 572-579; Ludewig, and Laman, 2004. PNAS USA. 101: 11529-11530; Moore and Freeman, 2006. Arterioscler Thromb Vasc Biol. 26: 1702-1711; Müller, R H., et al., 1996, International Journal of Pharmaceutics, 144: 115-121; Olbrich and Muller, 1999. Int J Pharm. 180: 31-39; Podrez, E A, et al., 2002. J Biol Chem. 277: 38517-38523 and 277: 38503-38516; Silverstein R L, 2009. Cleve Clin J Med. 76 Suppl 2: S27-S30; Sun et al., 2002. J. Org. Chem. 67: 3575-3584; Vaidyanathan and Walle, 2002. Drug Metab Dispos. 30, 897-903; Wang, S., et al., 2006. J Nutr Biochem. 17: 492-498; Warden, B A, et al., 2001. J Nutr. 131, 1731-1737; Watson, A D, et al., 1997. J Biol Chem. 272: 13597-13607; Wolfram, S., 2007. J Am Coll Nutr. 26: 373S-388S; Deng, Y., et al., 1998. J Org Chem. 63: 7789-7794; each of which is hereby incorporated by reference in its entirety.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

We claim:

1. A composition, comprising:
a plurality of nanoparticles comprising one or more oxidized phospholipids encapsulated within, adhered to a surface of, or integrated into the structure of the nanoparticles, wherein the one or more oxidized phospholipids target to atherosclerotic lesion sites.

2. The composition of claim 1, wherein the nanoparticles are selected from the group consisting of liposomes, polymerosomes, microspheres, micro-structured lipid carriers, nano-structured lipid carriers, high-density lipoproteins and a combination thereof.

3. The composition of claim 1, wherein the one or more oxidized phospholipids are selected from the group consisting of 1-palmitoyl-2-azelaoyl-sn-glycero-3-phosphocholine, 1-hexadecyl-2-azelaoyl-sn-glycero-3-phosphocholine, 1-palmitoyl-2-glutaroyl-sn-glycero-3-phosphocholine, 1-palmitoyl-2-(5-oxovaleroyl)-sn-glycero-3-phosphocholine, and 1-palmitoyl-2-(9-oxononanoyl)-sn-glycero-3-phosphocholine, an ester of lysophosphatidylcholine, an ester of 1-lysophosphatidylcholine (1-lysoPC), an ester of 2-lysophosphatidylcholine (2-lysoPC1-hexadecyl-2-acetoyl-sn-glycero-3-phosphocholine, 1-octadecyl-2-acetoyl-sn-glycero-3-phosphocholine, 1-hexadecyl-2-butyroyl-sn-glycero-3-phosphocholine, 1-octadecyl-2-butyroyl-sn-glycero-3-phosphocholine, 1-palmitoyl-2-acetoyl-sn-glycero-3-phosphocholine, 1-octadecenyl-2-acetoyl-sn-glycero-3-phosphocholine, 1-hexadecyl-2-(homogammalinolenoyl)-sn-glycero-3-phosphocholine, 1-hexadecyl-2-arachidonoyl-sn-glycero-3-phosphocholine, 1-hexadecyl-2-eicosapentaenoyl-sn-glycero-3-phosphocholine, 1-hexadecyl-2-docosahexaenoyl-sn-glycero-3-phosphocholine, 1-octadecyl-2-methyl-sn-glycero-3-phosphocholine, 1-hexadecyl-2-butenoyl-sn-glycero-3-phosphocholine, Lyso PAF C16, Lyso PAF C18, 1-O-1′-(Z)-hexadecenyl-2-[12-[(7-nitro-2-1,3-benzoxadiazol-4-yl)-amino]dodecanoyl]-sn-glycero-3-phosphocholine, 1-O-1-(Z)-hexadecenyl-2-oleoyl-sn-glycero-3-phosphocholine, 1-O-1-(Z)-hexadecenyl-2-arachidonoyl-sn-glycero-3-phosphocholine, 1-O-1′-(Z)-hexadecenyl-2-docosahexaenoyl-sn-glycero-3-phosphocholine, 1-O-1-(Z)-hexadecenyl-2-oleoyl-sn-glycero-3-phosphoethanolamine, 1-O-1′-(Z)-hexadecenyl-2-arachidonoyl-sn-glycero-3-phosphoethanolamine, 1-O-1′-(Z)-hexadecenyl-2-docosahexaenoyl-sn-glycero-3-phosphoethanolamine, and a combination thereof.

4. The composition of claim 1, wherein the one or more oxidized phospholipids are selected from the group consisting of 9-hydroxy-10-dodecenedioic acid esters of 2-lyso-PC (HDdiA-PC), 5-hydroxy-8-oxo-6-octenedioic acid esters of 2-lyso-PC(HOdiA-PC), 9-hydroxy-12-oxo-10-dodecenoic acid esters of 2-lyso-PC(HODA-PC), 5-hydroxy-8-oxo-6-octenoic acid esters of 2-lyso-PC(HOOA-PC), 9-keto-12-oxo-10-dodecenoic acid esters of 2-lyso-PC (KODA-PC), 5-keto-8-oxo-6-octenoic acid esters of 2-lyso-PC (KOOA-PC), 9-keto-10-dodecendioic acid esters of 2-lyso-PC (KDdiA-PC), and 5-keto-6-octendioic acid esters of 2-lyso-PC (KOdiA-PC), 5-oxovaleric acid esters of 2-lyso-PC (OV-PC), and 9-oxononanoic acid esters of 2-lyso-PC(ON-PC), 1-palmitoyl-2-(5-oxovaleroyl)-phosphatidylcholine (POVPC), and a combination thereof.

5. The composition of claim 1, wherein the one or more oxidized phospholipids comprise KDdiA-PC.

6. The composition of claim 1, wherein the nanoparticles consist of the one or more oxidized phospholipids selected from the group consisting of 9-hydroxy-10-dodecenedioic acid esters of 2-lyso-PC (HDdiA-PC), 5-hydroxy-8-oxo-6-octenedioic acid esters of 2-lyso-PC (HOdiA-PC), 9-hydroxy-12-oxo-10-dodecenoic acid esters of 2-lyso-PC(HODA-PC), 5-hydroxy-8-oxo-6-octenoic acid esters of 2-lyso-PC(HOOA-PC), 9-keto-12-oxo-10-dodecenoic acid esters of 2-lyso-PC (KODA-PC), 5-keto-8-oxo-6-octenoic acid esters of 2-lyso-PC (KOOA-PC), 9-keto-10-dodecendioic acid esters of 2-lyso-PC (KDdiA-PC), and 5-keto-6-octendioic acid esters of 2-lyso-PC (KOdiA-PC), 5-oxovaleric acid esters of 2-lyso-PC (OV-PC), and 9-oxononanoic acid esters of 2-lyso-PC (ON-PC), 1-palmitoyl-2-(5-oxovaleroyl)-phosphatidylcholine (POVPC), and a combination thereof.

7. The composition of claim 1, wherein the nanoparticles comprise one or more molecules selected from the group consisting of albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(-hydroxyvalerate), poly(D,L-lactide-co-glycolide), poly(1-lactide-co-glycolide), poly(-hydroxybutyrate), poly(-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, apolioprotein A1, apolioprotein A2, other apolioproteins and a combination thereof.

8. The composition of claim 1, wherein the nanoparticles comprise:
one or more molecules selected from the group consisting of gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, and a combination thereof;
a biodegradable polymer comprises PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride), and a combination thereof or
phospholipids, cholesterol, sphingolipids, ceramides, plant sterol, cholesterol or hapten-conjugated lipids.

9. The composition of claim 1, wherein the nanoparticles further comprises:
an additional targeting ligand, encapsulated within, adhered to a surface of, or integrated into the structure of the nanoparticles, wherein the additional targeting ligand also targets an atherosclerotic lesion site.

10. The composition of claim 9, wherein each of the nanoparticles comprises both the one or more oxidized phospholipids and the additional targeting ligand.

11. The composition of claim 9, wherein the additional targeting ligand is an antibody or an antibody fragment that recognizes a protein at or near an atherosclerotic lesion site, wherein the antibody or an antibody fragment is reactive to one selected from the group consisting of oxidized LDL, scavenger receptor A (the first OxLDL receptor to be characterized and cloned, CD36, CD68, Lectin-like oxidized LDL receptor-1 (LOX-1), SR-A1, SR-B1, and a combination thereof.

12. The composition of claim 1, further comprising:
one or more bioactive agents encapsulated within, adhered to a surface of, or integrated into the structure of the nanoparticles, wherein the one or more bioactive agents are delivered to or near an atherosclerotic lesion site, wherein the one or more bioactive agents comprise a first bioactive agent that provides an indicium for the presence of the atherosclerotic lesion site.

13. The composition of claim 12, wherein the one or more bioactive agents further comprise:
a second bioactive agent that exhibits a therapeutic effect on the atherosclerotic lesion site.

14. The composition of claim 12, wherein the indicium is a fluorescent signal emitted upon binding of the first bioactive agent to or near the atherosclerotic lesion site.

15. The composition of claim 1, further comprising:
an adjuvant or a pharmaceutically compatible carrier.

16. A method of preventing, diagnosing and/or treating atherosclerosis in a patient, comprising:
administering an effective amount of a composition, to or near a known or suspected atherosclerotic lesion site, wherein the composition comprises:
a plurality of nanoparticles comprising one or more oxidized phospholipids encapsulated within, adhered to a surface of, or integrated into the structure of the nanoparticles, wherein the one or more oxidized phospholipids target the atherosclerotic lesion site.

17. The method of claim 16, wherein the administering step comprises intraarterial delivery of the nanoparticles.

18. The method of claim 16, wherein intraarterial or intravenous delivery comprises using a catheter or direct injection.

19. A method of making nanoparticles, comprising a phase inversion method step, wherein the nanoparticles comprising one or more oxidized phospholipids encapsulated within, adhered to a surface of, or integrated into the structure of the nanoparticles, wherein the one or more oxidized phospholipids target a atherosclerotic lesion site.
